(19) United States
(12) Patent Application Publication
(10) Pub. No.: US 2022/0304549 A1
(43) Pub. Date: Sep. 29, 2022

(54) ENDOSCOPE

(71) Applicant: Endoscopy Development Company, LLC, Maryland Heights, MO (US)

(72) Inventor: Kazuo Iijima, Yokohama (JP)

(73) Assignee: Endoscopy Development Company, LLC, Maryland Heights, MO (US)

(21) Appl. No.: 17/704,475

(22) Filed: Mar. 25, 2022

Related U.S. Application Data
(60) Provisional application No. 63/166,021, filed on Mar. 25, 2021.

(51) Int. Cl. A61B 1/005; A61B 1/00; A61B 1/07

(52) U.S. Cl. CPC A61B 1/0052; A61B 1/005; A61B 1/00103; A61B 1/00114; A61B 1/07; A61B 1/00124; A61B 1/0057

(57) ABSTRACT

An endoscope includes a control body, an insertion tube, a bending section, and an angulation drive assembly. The control body includes a first body and a second body. The first body includes a first stepped interface having a first upper surface, a second upper surface, and a first intermediate surface. The second body is coupled to the first body and includes a second stepped interface having a first lower surface, a second lower surface, and a second intermediate surface. The bending section is secured to the insertion tube. The angulation drive assembly is coupled to the bending section and is operable to articulate the bending section. When the first body and the second body are connected to each other, the first lower surface abuts against the first upper surface, the second lower surface abuts against the second upper surface, and the first intermediate surface abuts against the second intermediate surface.

### CROSS-REFERENCE TO RELATED APPLICATIONS

The present application claims the benefit of U.S. Provisional Application No. 63/166,021, titled “ENDOSCOPE” and filed Mar. 25, 2021. The disclosure of the above application is incorporated herein by reference.

### FIELD

The present disclosure relates to an endoscope.

### BACKGROUND

The statements in this section merely provide background information related to the present disclosure and may not constitute prior art.

Endoscopes are used in a wide variety of medical procedures to visualize internal cavities within the human body, for example, during diagnostic or therapeutic procedures. Some endoscopes include a reusable body and a disposable body that is removably connected to the reusable body. A flexible insertion tube is coupled to an end of the disposable body and is inserted into the internal cavity of the human body. Such endoscopes are designed to dispose the disposable body and the insertion tube after use, thereby reducing the risk of infectious disease transmission. Further, in such endoscopes, an angulation assembly that includes wires is configured to articulate a bending section coupled to a distal end of the insertion tube. The wires may become loose overtime, thereby calling for regular maintenance.

The bending section of conventional endoscopes may include a series of stacked links that are movably coupled to each other. When wires articulate the bending section, the links may move in a twisting motion, which adversely effects the articulation of the bending section.

The present disclosure addresses these and other issues associated with endoscopes.

### SUMMARY

This section provides a general summary of the disclosure and is not a comprehensive disclosure of its full scope or all of its features.

In one form, the present disclosure provides an endoscope including a control body, an insertion tube, a bending section, and an angulation drive assembly. The control body includes a reusable first body and a disposable second body. The reusable first body includes a first stepped interface. the first stepped interface has a first upper surface, a second upper surface, and a first lateral surface positioned between the first upper surface and the second upper surface. The disposable second body is removably coupled to the first body and includes a second stepped interface. The second stepped interface has a first lower surface, a second lower surface, and a second lateral surface positioned between the first lower surface and the second lower surface. The insertion tube is coupled to the control body. The bending section is secured to a distal end of the insertion tube. The angulation drive assembly is coupled to the bending section and is operable to articulate the bending section. When the reusable first body and the disposable second body are connected to each other, the first lower surface abuts against the first upper surface, the second lower surface abuts against the second upper surface, and the first lateral surface abuts against the second lateral surface.

In variations of the endoscope of the above paragraph, which may be implemented individually or in any combination: the first lateral surface and the second lateral surface extend in a longitudinal direction of the endoscope; the first and second lateral surfaces are flat; the first and second upper surfaces have a semi-circular shape, the first and second lower surfaces have a semi-circular shape; the first and second upper surfaces extend orthogonal to the first lateral surface, the first and second lower surfaces extend orthogonal to the second lateral surface; a spring-loaded connecting member extends from the first upper surface of the reusable first body, the spring-loaded connecting member is configured to be received in a pocket formed in the first lower surface of the disposable second body to inhibit the reusable first body and the disposable second body from separating from each other in a lateral direction; the angulation drive assembly includes a first pulley and a second pulley, the first pulley is associated with the reusable first body and defines a first rotational axis, the second pulley is associated with the disposable second body and defines a second rotational axis, the second pulley is coupled to the first pulley such that rotation of the first pulley is transmitted to the second pulley, the first pulley and second pulley are coupled to each other at a location where the first lateral surface abuts against the second lateral surface; a first electrical coupling is associated with the reusable first body and a second electrical coupling is associated with the disposable second body, the first and second electrical couplings are electrically coupled to each other at a location where the first lateral surface abuts against the second lateral surface; a first electrical coupling is associated with the reusable first body and a second electrical coupling is associated with the disposable second body, the first and second electrical couplings are electrically coupled to each other at a location between the spring-loaded connecting member and the first and second pulleys, the reusable first body includes a first end wall that defines the first upper surface, the disposable second body includes a second end wall that defines the first lower surface, at least one window formed in one of the first end wall or the second end wall, a first fiber bundle is at least partially disposed within the reusable first body and has a first end electrically connected to an illumination source and a second end adjacent to the at least one window, a second fiber bundle is at least partially disposed within the disposable second body and is spaced apart from the first fiber bundle, the second fiber bundle has a third end adjacent to the at least one window and a fourth end associated with the insertion tube, the third end of the second fiber bundle being coaxial with the second end of the first fiber bundle so that light from the illumination source is transmitted from the first fiber bundle to the second fiber bundle, the at least one window is formed in the first end wall; the endoscope comprises a first window formed in the first end wall and a second window formed in the second end wall; the first fiber bundle includes a first diameter and the second fiber bundle includes a second diameter, the first diameter is greater than the second diameter; the at least one window is made of a transparent material; a female electrical connector is formed in the first lateral surface of the reusable first body; a male electrical connector extends from the second lateral surface of the disposable second body; the male electrical connector is configured to be received in the electrical female connector to electrically couple the female electrical connector and the male electrical connector to each other; the handle assembly includes a connecting member and a knob, the connecting member is rotationally fixed to a pulley of the angulation drive assembly, the knob is removably coupled to the connecting member. When the knob is coupled to the connecting member, rotation of the knob causes corresponding rotation of the connecting member and the pulley of the angulation drive assembly; the knob includes a boss extending therefrom and the connecting member includes a plurality of apertures formed therein, the boss is configured to be received in one aperture of the plurality of apertures to position the knob with respect to the connecting member; the knob is coupled to the connecting member at a location external to the control body; a sealing member is disposed within the reusable first body and is received in an annular groove of the connecting member; an attachment mechanism including a first attachment member associated with one of the reusable first body and the disposable second body and a second attachment member associated with the other of the reusable first body and the disposable second body, the first attachment member snaps into engagement with the second attachment member; the first attachment member is a flexible tab secured to the one of the reusable first body and the disposable second body and the second attachment member is a recess formed in the other of the reusable first body and the disposable second body; and the attachment mechanism secures the first reusable body and the second disposable body to each other at a location diametrically opposite of the spring-loaded connecting member.

In another form, the present disclosure discloses an endoscope including a control body, an insertion tube, a bending section, and an angulation drive assembly. The control body includes a first body and a second body removably coupled to the first body. The insertion tube is coupled to the control body. The bending section is secured to a distal end of the insertion tube. The angulation drive assembly is coupled to the bending section and is operable to articulate the bending section. The angulation drive assembly includes a first pulley device and a second pulley device. The first pulley device is associated with the first body and includes a first pulley and a first transmission element. The first pulley defines a first rotational axis. The second pulley device is associated with the second body and includes a second pulley and a second transmission element operably connected to the bending section. The second pulley is coupled to the first pulley such that rotation of the first pulley causes rotation of the second pulley. The second pulley also defines a second rotational axis. The first rotational axis of the first pulley and the second rotational axis of the second pulley are aligned with each other.

In variations of the endoscope of the above paragraph, which may be implemented individually or in any combination: the first body is reusable and the second body is disposable; the first transmission element is a belt and the second transmission element is one or more wires; the first transmission element is made of a first material and the second transmission element is made of a second material, the first material has a greater strength than the second material; the first pulley includes first teeth, the first pulley device further includes a knob pulley that includes second teeth, the first transmission element transmits rotational motion of the knob pulley to the first pulley, there are at least twice as many second teeth than first teeth; the handle assembly includes a connecting member and a knob, the connecting member is rotationally fixed to the knob pulley, the knob is removably coupled to the connecting member at a location external to the control body, when the knob is coupled to the connecting member, rotation of the knob causes corresponding rotation of the connecting member and the knob pulley; the first pulley includes a central recess formed therein, the second pulley includes a central peg that corresponds to and is disposed within the central recess such that the first pulley and second pulley are rotationally fixed to each other; the second pulley device is located external relative to the first body; the first pulley comprises a first magnet and the second pulley comprises a second magnet, and wherein the first magnet and the second magnet are attracted to each other such that the second pulley and the first pulley are rotationally secured to each other when the first and second bodies are coupled to each other; and when the first and second bodies are coupled to each other, the first and second bodies define a compartment, the second magnet disposed within the compartment.

In yet another form, the present disclosure discloses an endoscope including a control body, an insertion tube, a bending section, an angulation drive assembly, and a handle assembly. The insertion tube is coupled to the control body. The bending section is secured to a distal end of the insertion tube. The angulation drive assembly is coupled to the bending section and is operable to articulate the bending section. The handle assembly includes a connecting member and a knob. The connecting member is at least partially disposed within the control body and is rotationally fixed to a pulley of the angulation drive assembly. The connecting member includes a plurality of apertures formed in a first connecting surface. The knob is removably coupled to the connecting member and includes a boss extending from a second connecting surface. The boss is configured to be received in one aperture of the plurality of apertures to position the knob with respect to the connecting member. When the knob is coupled to the connecting member, rotation of the knob causes corresponding rotation of the pulley of the angulation drive assembly.

In variations of the endoscope of the above paragraph, which may be implemented individually or in any combination: the knob is coupled to the connecting member at a location external to the control body; a sealing member is disposed within the control body and is received in an annular groove of the connecting member, the sealing member is sealingly engaged with the control body to inhibit fluids and debris from entering into the control body; the first connecting surface and the second connecting surface are flat; the plurality of apertures are circumferentially spaced apart around the first connecting surface.

In yet another form, the present disclosure discloses an endoscope including a control body, an insertion tube, a bending section, and an angulation drive assembly. The insertion tube is coupled to the control body. The bending section is secured to a distal end of the insertion tube. The angulation drive assembly includes a transmission element coupled to the bending section. The transmission element is operable to articulate the bending section. The bending section includes a series of stacked links movably coupled to each other. Each link includes a body, a pair of reinforcement walls, and a pair of protrusions. The body includes an outer surface and an inner surface. The body also defines a pair of recesses formed near a first end of the body. The pair of reinforcement walls are coupled to the body proximate the pair of recesses. The pair of protrusions extend from a second end of the body. Each protrusion is configured to be received in a respective recess of the pair of recesses such that the protrusion abuts against a respective reinforcement wall.

In variations of the endoscope of the above paragraph, which may be implemented individually or in any combination: the pair of protrusions are diametrically opposed to each other and the pair of recesses are diametrically opposed to each other, each reinforcement wall defines an engagement surface, and wherein the engagement surface faces away from the inner surface of the body; each recess of the pair of recesses further includes an arcuate surface. Each protrusion includes an outer end surface that corresponds to the arcuate surface; each recess of the pair of recesses further includes an arcuate surface; the body of each link includes a channel extending therethrough. The transmission element extends through the channel.

In yet another form, the present disclosure discloses an articulation mechanism for use in a medical device. The articulation mechanism includes a series of stack links and at least one transmission element. The stacked links are movably coupled to each other and includes a body, a pair of reinforcement walls and a pair of protrusions. The body includes an outer surface and an inner surface. The body also defines a pair of recesses formed near a first end of the body. The pair of reinforcement walls are coupled to the body proximate the pair of recesses. The pair of protrusions extend from a second end of the body. Each protrusion is configured to be received in a respective recess of the pair of recesses such that the protrusion abuts against a respective reinforcement wall. At least one transmission element extends through the series of stacked links and is configured to articulate the series stacked links. The at least one transmission element also holds the series of stacked links together.

Further areas of applicability will become apparent from the description provided herein. It should be understood that the description and specific examples are intended for purposes of illustration only and are not intended to limit the scope of the present disclosure.

### DRAWINGS

In order that the disclosure may be well understood, there will now be described various forms thereof, given by way of example, reference being made to the accompanying drawings, in which:

FIG. 1 is a perspective view of an endoscope constructed according to the teachings of the present disclosure;

FIG. 2 is a perspective view of a control body of the endoscope of FIG. 1;

FIG. 3A is an exploded side view of a portion of the control body of FIG. 2;

FIG. 3B is a side view of the control body of FIG. 2 and a detail view of a portion;

FIG. 3C is an enlarged view of the control body of FIG. 3B illustrating fiber bundles according to the teachings of the present disclosure;

FIG. 4 is an exploded perspective view of a portion of the control body of FIG. 2;

FIG. 5 is another exploded perspective view of a portion of the control body of FIG. 2;

FIG. 6 is a perspective view of a reusable first body of the control body of FIG. 2 showing a first pulley device of an angulation drive assembly of the endoscope of FIG. 1 disposed within the reusable first body with a lateral wall of the reusable first body removed for clarity;

FIG. 7 is a perspective view of a disposable second body of the control body of FIG. 2 showing a second pulley device of the angulation drive assembly of the endoscope of FIG. 1 disposed within the disposable second body with a lateral wall of the disposable second body removed for clarity;

FIG. 8 is an exploded perspective view of the control body of FIG. 2 with a drive pulley of the first pulley device of FIG. 6 being connected to a driven pulley of the second pulley device of FIG. 7;

FIG. 9 is an exploded perspective view of a handle assembly of the endoscope of FIG. 1;

FIG. 10 is another exploded perspective view of the handle assembly;

FIG. 11 is a perspective view of the reusable first body showing a first fiber bundle disposed within the reusable first body with the lateral wall of the reusable first body removed for clarity;

FIG. 12 is a perspective view of the disposable second body showing a second fiber bundle disposed within the disposable second body with the lateral wall of the disposable second body removed for clarity;

FIG. 13 is a perspective view of the disposable second body showing the second fiber bundle disposed within the disposable second body;

FIG. 14 is a perspective view of a bending section of the endoscope of FIG. 1;

FIG. 15 is a perspective view of a portion of the bending section of FIG. 14;

FIG. 16 is a perspective view of one link of the bending section of FIG. 14;

FIG. 17 is another perspective view of one link of the bending section of FIG. 14;

FIG. 18 is a cross-sectional view of a portion of the bending section of FIG. 14;

FIG. 19 is a perspective view an alternate endoscope constructed according to the teachings of the present disclosure;

FIG. 20 is a partial perspective view of the endoscope of FIG. 19 with the attachment feature in a release position;

FIG. 21 is a partial perspective view of the endoscope of FIG. 19 with an attachment feature in a secured position;

FIG. 22 is a perspective view an alternate endoscope constructed according to the teachings of the present disclosure;

FIG. 23 is a partial perspective view of the endoscope of FIG. 22 with an attachment member secured to a reusable body of the endoscope;

FIG. 24 is a partial perspective view of a disposable body of the endoscope of FIG. 22 comprising an attachment member;

FIG. 25 is a perspective view another alternate endoscope constructed according to the teachings of the present disclosure;

FIG. 26 is a side view of the endoscope of FIG. 25;

FIG. 27 is a side view of a reusable body of the endoscope of FIG. 25;

FIG. 28 is a perspective view of the reusable body of the endoscope of FIG. 25 shown in hidden lines;

FIG. 29 is a perspective view of the reusable body of the endoscope of FIG. 25;

FIG. 30 is a perspective view of a disposable body of the endoscope of FIG. 25;

FIG. 31 is a perspective view of an angulation drive assembly that is configured to be disposed within the reusable body of the endoscope of FIG. 25;

FIG. 32 is a side view of the angulation drive assembly of FIG. 31;

FIG. 33 is a perspective view an alternate endoscope constructed according to the teachings of the present disclosure;

FIG. 34 is a perspective view of a reusable body of the endoscope of FIG. 33; and

FIG. 35 is a perspective view of a disposable body of the endoscope of FIG. 33.

The drawings described herein are for illustration purposes only and are not intended to limit the scope of the present disclosure in any way.

### DETAILED DESCRIPTION

The following description is merely exemplary in nature and is not intended to limit the present disclosure, application, or uses. It should be understood that throughout the drawings, corresponding reference numerals indicate like or corresponding parts and features.

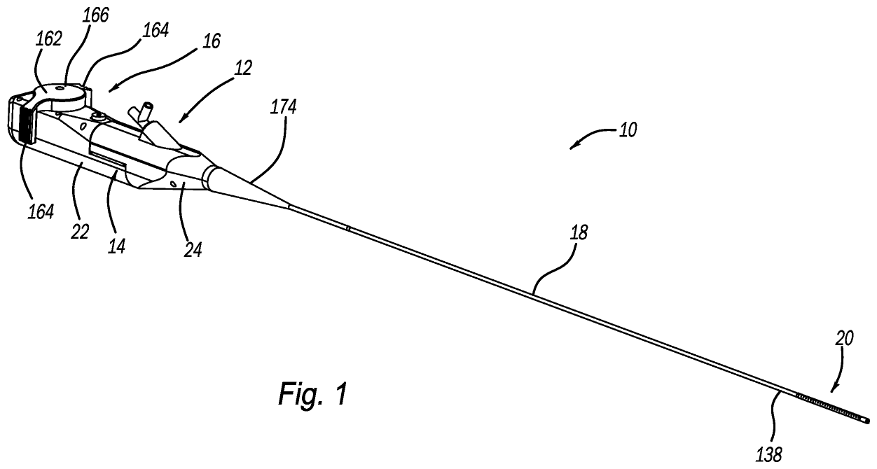

As shown in FIG. 1, an endoscope 10 is provided. The endoscope 10 includes a control body 12, an angulation drive assembly 14, a handle assembly 16, a flexible insertion tube 18, and a bending section or articulation mechanism 20. The control body 12 is configured to be grasped or held by a user and may be a generally cylindrical shape as shown.

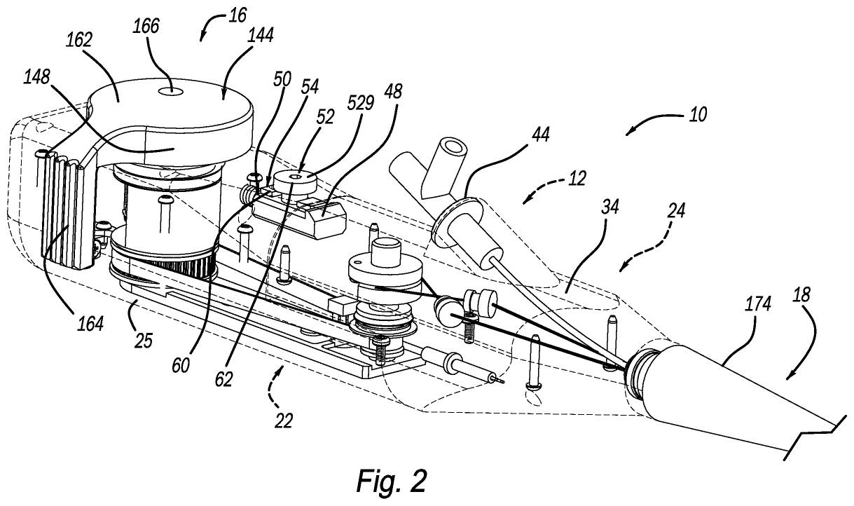

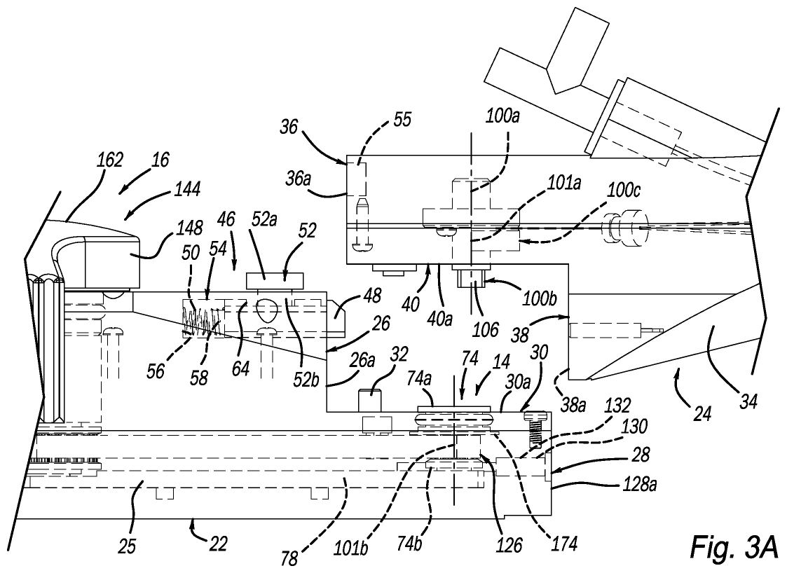

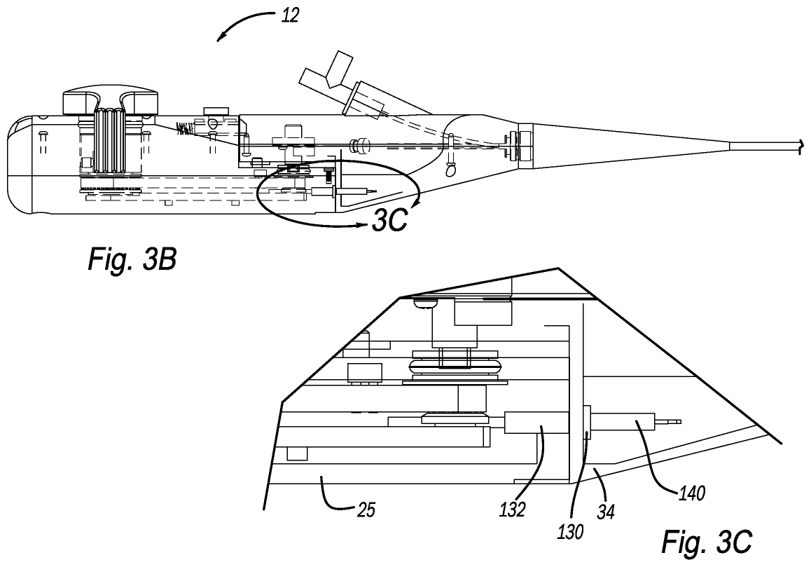

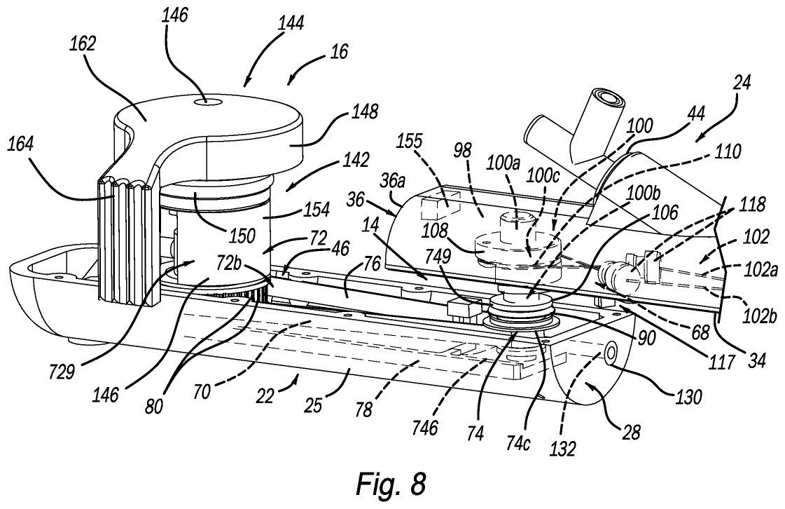

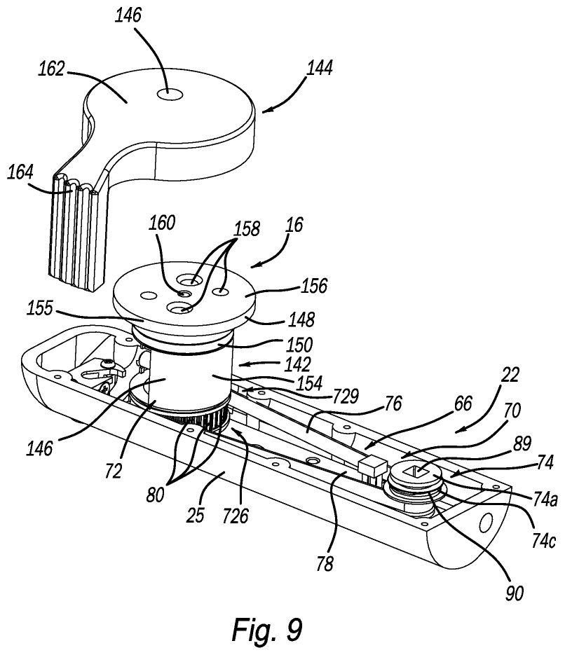

With reference to FIGS. 2-13, the control body 12 includes a reusable first body 22 (FIGS. 1-6 and 8-11) and a disposable second body 24 (FIGS. 1-5, 7, 8, 12, and 13). The reusable first body 22 includes a casing 25, a first upper wall 26, a second upper wall 28 and a lateral or intermediate wall 30. The first upper wall 26 is fixed to the casing 25 and has semi-circular shape. The first upper wall 26 extends in a transverse direction of the endoscope 10 and includes a flat upper surface 26a. Similarly, the second upper wall 28 is fixed to the casing 25 and has semi-circular shape. The second upper wall 28 also extends in a transverse direction of the endoscope 10 and includes a flat upper surface 28a. The lateral wall 30 is removably coupled to the casing 25 via fasteners, for example, and extends in a longitudinal direction of the endoscope 10. The lateral wall 30 extends orthogonal to the first and second upper walls 26, 28 and includes a flat lateral or intermediate surface 30a. In some configurations, the lateral wall 30 may extend in an obtuse angle with respect to the first and second upper walls 26, 28. As shown in FIGS. 3-5, a plurality of circular-shaped bosses 32 extend from the lateral surface 30a. The bosses 32 are spaced apart from each other. In some configurations, the bosses 32 may be triangular, square, pentagonal, or any other suitable shape. The surfaces 26a, 28a, 30a of the walls 26, 28, 30, respectively, cooperate with each other to form a stepped interface.

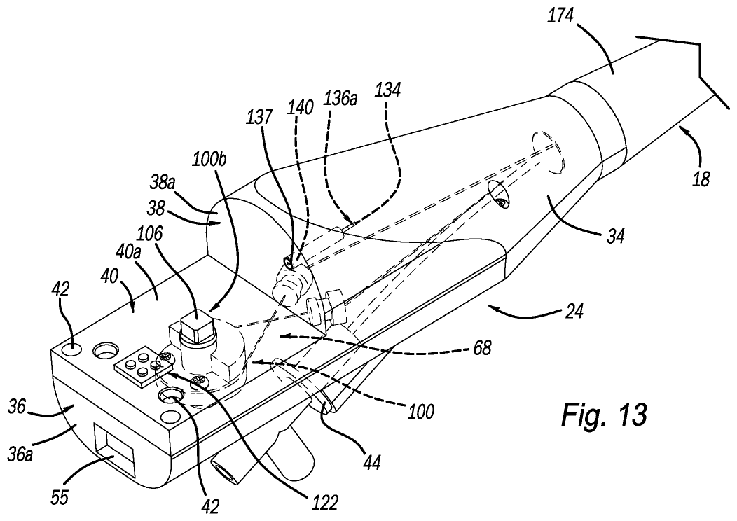

The disposable second body 24 is removably coupled to the reusable first body 22 and includes a casing 34, a first lower wall 36, a second lower wall 38 and a lateral or intermediate wall 40. The first lower wall 36 is fixed to the casing 34 and has semi-circular shape. The first lower wall 36 extends in a transverse direction of the endoscope 10 and includes a flat lower surface 36a. Similarly, the second lower wall 38 is fixed to the casing 34 and has semi-circular shape. The second lower wall 38 also extends in a transverse direction of the endoscope 10 and includes a flat lower surface 38a. The lateral wall 40 is removably coupled to the casing 34 via fasteners, for example, and extends in a longitudinal direction of the endoscope 10. The lateral wall 40 extends orthogonal to the first and second lower walls 36, 38 and includes a flat lateral or intermediate surface 40a. In some configurations, the lateral wall 40 may extend in an obtuse angle with respect to the first and second lower walls 36, 38. As shown in FIGS. 5 and 13, a plurality of openings 42 may be formed in the lateral surface 40a. The openings 42 correspond to the bosses 32 of the reusable first body 22 and are configured to receive a respective boss 32. In this way, when the reusable first body 22 and the disposable second body 24 are connected to each other, the disposable second body 24 is inhibited from detaching from the reusable first body 22 in the longitudinal direction. The surfaces 36a, 38a, 40a of the walls 36, 38, 40, respectively, cooperate with each other to form a stepped interface.

When the reusable first body 22 and the disposable second body 24 are connected to each other, the lower surface 36a abuts against the upper surface 26a, the lower surface 38a abuts against the upper surface 28a, and the lateral surface 40a abuts against the lateral surface 30a. One or more ports 44 may be coupled to the disposable second body 24 and may be in communication with one or more lumens (not shown) extending through the insertion tube 18 and the bending section 20 so as to allow for a tool and/or fluid to pass through the ports 44 and into the lumens. Additionally or alternatively, one or more ports (not shown) may be coupled to the reusable first body 22 and in communication with one or more lumens extending through the insertion tube 18 and the bending section 20.

Although the present disclosure describes the first body 22 as reusable and the second body 24 as disposable, in some configurations, both the first body 22 and the second body 24 may be reusable or both of the first body 22 and the second body 24 may be disposable.

With reference to FIGS. 2, 3, 4 and 10, a coupling assembly 46 is associated with the reusable first body 22 and includes an attachment member 48, a biasing member 50 (FIGS. 2 and 3) and a locking member 52. The attachment member 48 has a hook shape and is disposed within a pocket 54 formed in the upper wall 26 of the reusable first body 22. The attachment member 48 is movable between an unlocked position in which the attachment member 48 is disengaged with the disposable second body 24 (the attachment member 48 is entirely received in the pocket 54) and a locked position in which the attachment member 48 is engaged with the disposable second body 24 (the attachment member 48 extends from the pocket 54 and into a rectangular-shaped recess 55 formed in the lower surface 36a of the disposable second body 24). When the attachment member 48 is in the unlocked position, the reusable first body 22 and the disposable second body 24 may detach from each other in the lateral direction (in a direction perpendicular to the longitudinal direction of the endoscope 10). When the attachment member 48 is engaged with the disposable second body 24, the reusable first body 22 and the disposable second body 24 are coupled together and thus inhibited from detaching from each other in the lateral direction.

The biasing member 50 such as a spring is disposed within the pocket 54 in the reusable first body 22. As shown in FIG. 3, the biasing member 50 engages a surface 56 of the pocket 54 at first end and is secured to a projection 58 of the attachment member 48 at a second end. In this way, the biasing member 50 biases the attachment member 48 toward the locked position. The locking member 52 extends through an opening 60 in the casing 25 and is configured to rotate between a locked state and an unlocked state. When the locking member 52 is in the locked state, the attachment member 48 is in the locked position. When the locking member 52 is in the unlocked state, the attachment member 48 is in the unlocked position. The locking member 52 includes a locking end 52a and a cam end 52b (FIG. 3). The locking end 52a is circular-shaped and is located externally relative to the reusable first body 22. The locking end 52a includes an opening 62 that is configured to receive a tool (not shown) such as a hex key. The opening 62 shown in the figures is hexagonal-shaped, however, in some configurations, the shape of the opening 62 may be pentagonal, square, octagonal or any other suitable shape that is allowed to be operable using a tool. The cam end 52b extends from the locking end 52a and is received in a recess 64 (FIG. 3) formed in the attachment member 48. In this way, when the tool rotates the locking member 52 from the unlocked state toward the locked state, for example, the cam end 52b rotates in the recess 64, thereby causing the attachment member 48 to move in a longitudinal direction from the unlocked position toward the locked position.

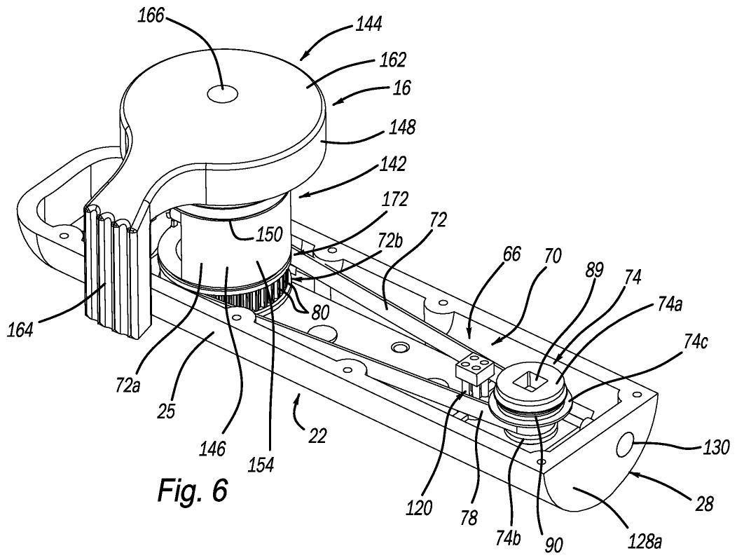

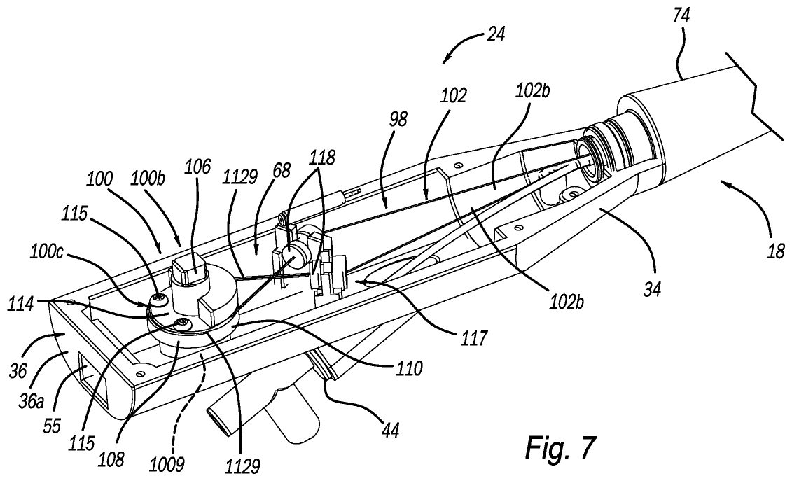

The angulation drive assembly 14 is coupled to the bending section 20 and is operable to articulate the bending section 20. The angulation drive assembly 14 includes a first pulley device 66 (best shown in FIGS. 6, 8, 9, and 11) and a second pulley device 68 (best shown in FIGS. 7, 8 and 12) operatively connected to the first pulley device 66. The first pulley device 66 is associated with the reusable first body 22. For example, the first pulley device 66 is at least partially disposed within a cavity 70 of the reusable first body 22 (the cavity 70 is formed by the casing 25 of the reusable first body 22). The first pulley device 66 includes a knob pulley 72, a drive pulley 74, and a transmission element 76. The knob pulley 72 is rotatably engaged to the casing 25 and is rotationally fixed to the handle assembly 16. In some configurations, as shown in the figures, the knob pulley 72 is rotatably engaged to a plate 78 that is disposed within and secured to the casing 25 of the reusable first body 22. In one example, the plate 78 may be secured to the casing 25 via fasteners such as screws, bolt, and rivets, among others. In another example, the plate 78 may be secured to locking features (e.g., grooves) formed in the casing 25 of the reusable first body 22. The plate 78 includes a first protrusion (not shown) positioned at a first end thereof that is received a central opening (not shown) in the knob pulley 72.

As shown in FIGS. 6, 8, 9, and 11, the knob pulley 72 is generally cylindrical shape and includes a handle portion 72a and a transmission element portion 72b. The handle portion 72a is rotationally fixed to the handle assembly 16 such that rotation of the handle assembly 16 causes corresponding rotation of the knob pulley 72. The transmission element portion 72b extends from the handle portion 72a and includes a plurality of teeth 80 formed around an outer circumferential surface thereof. A diameter of an outer circumferential surface of the handle portion 72a is greater than a diameter of the outer circumferential surface of the transmission element portion 72b.

The drive pulley 74 is operatively connected to the knob pulley 72 and rotationally fixed to the second pulley device 68 so that rotation of the drive pulley 74 operates the second pulley device 68. The drive pulley 74 is also rotatably engaged to the plate 78. With reference to FIGS. 3, 4, 6, 8, 9, and 11, the drive pulley 74 includes a cylindrical-shaped sealing portion 74a, a cylindrical-shaped transmission element portion 74b, and a flange portion 74c positioned between the sealing portion 74a and the transmission element portion 74b. The sealing portion 74a extends from a first side of the flange portion 74c and is received in a circular opening formed in the lateral wall 30 of the reusable first body 22 so that the lateral wall 30 rotatably supports the drive pulley 74. The sealing portion 74a extends past the lateral wall 30 away from the cavity 70 and includes an annular groove and a locking groove 89. A sealing member 90 (e.g., an O-ring) is received in the annular groove and is sealingly engaged with an inner circumferential surface of the lateral wall 30. In this way, fluid and debris are inhibited from entering into the cavity 70 of the reusable first body 22. The locking groove 89 is generally a square shaped and is formed in a central part of the sealing portion 74a.

The transmission element portion 74b extends from a second side of the flange portion 74c and includes a plurality of teeth formed around an outer circumferential surface thereof. There are at least twice as many teeth 80 on the knob pulley 72 as the teeth on the transmission element portion 74b of the drive pulley 74. In this way, a user may apply less force to the handle assembly 16 to operate the angulation drive assembly 14. The plate 78 includes a second protrusion (not shown) positioned at a second end thereof that is received a central opening (not shown) in the transmission element portion 74b. The flange portion 74c abuts against a flat lateral surface (not shown) of the lateral wall 30, which opposes the lateral surface 30a of the lateral wall 30.

The transmission element 76 may be a timing belt, for example, and may include teeth (not shown) on an inner surface thereof. The teeth of the transmission element 76 are meshingly engaged with teeth 80 of the knob pulley 72 and with teeth of the drive pulley 74. In this way, rotation of the knob pulley 72 via the handle assembly 16 is transmitted to the drive pulley 74 via the transmission element 76, thereby causing corresponding rotation of the drive pulley 74.

The second pulley device 68 is associated with the disposable second body 24. For example, the second pulley device 68 is at least partially disposed within a cavity 98 of the casing 34 and includes a driven pulley 100 and a transmission element 102. The driven pulley 100 includes a rotational axis 101a that is coaxial with a rotational axis 101b of the drive pulley 74 (FIG. 3), which provides for the transmission element 76 being tensioned bisymmetrically. The driven pulley 100 is also rotatably engaged to the casing 34 and rotationally fixed to the drive pulley 74. In this way, rotation of the drive pulley 74 causes rotation of the driven pulley 100.

With reference to FIGS. 3-5, 7, 8, 12, and 13, the driven pulley 100 includes a first peg 100a, a second peg 100b and a transmission element portion 100c positioned between the first peg 100a and the second peg 100b. The first peg 100a extends from a first side of the transmission element portion 100c. The first peg 100a has a cylindrical shape and is disposed in a corresponding opening formed in the casing 34. In this way, the casing 34 rotatably supports the driven pulley 100.

The second peg 100b extends from a second side of the transmission element portion 100c. The second peg 100b has an end 106 that is generally a square shape and is disposed in the locking groove 89 formed in the drive pulley 74, thereby rotationally fixing the drive pulley 74 and the driven pulley 100 to each other. It should be understood that the square shape of the second peg 100b is merely exemplary and that other shapes may also be employed, such as by way of example rectangular or any other polygonal shape. Correspondingly, the locking groove 89 would define a mating geometry to any such alternate shape for the second peg 100b. The transmission element portion 100c includes a circumferential surface 108 having an arcuate groove 110 (FIGS. 7, 8, and 12) formed therein.

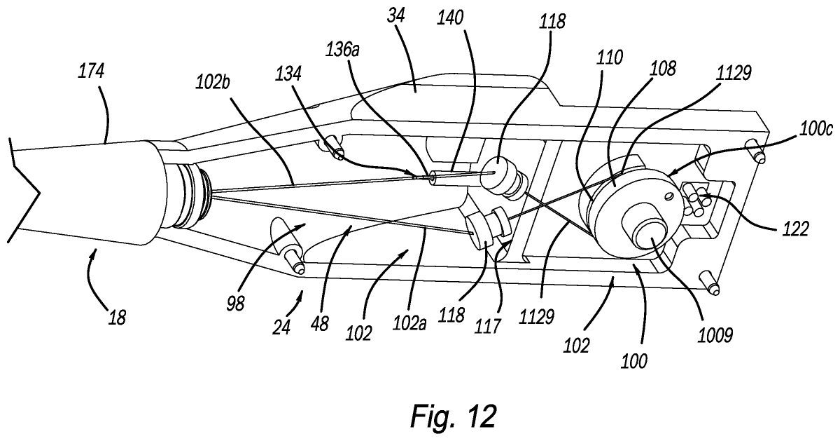

The transmission element 102 may be a pair of wires 102a, 102b that are partially received in the groove 110 formed in the transmission element portion 100c and that extend through the insertion tube 18 and the bending section 20. A first end 112a of each wire 102a, 102b is secured to a surface 114 of the transmission element portion 100c via a fastener 115 and a second end (not shown) of each wire 102a, 102b is secured to the bending section 20. In this way, when the driven pulley 100 rotates, as described above, the wires 102a, 102b operate to articulate the bending section 20. A partition 117 (best shown in FIGS. 7 and 12) is disposed within the cavity 98 of the casing 34 and is fixed to an inner surface of the casing 34. A pair of spacers 118 are secured to the partition 117 and are spaced apart from each other. Each wire 102a, 102b extends through a respective spacer 118 so that the wires 102a, 102b maintain a distance apart from each other. Although the transmission element 102 is shown in the figures as having two wires, the transmission element 102 may have one wire or more than two wires. The material of the transmission element 76 may be stronger than the material of the transmission element 102.

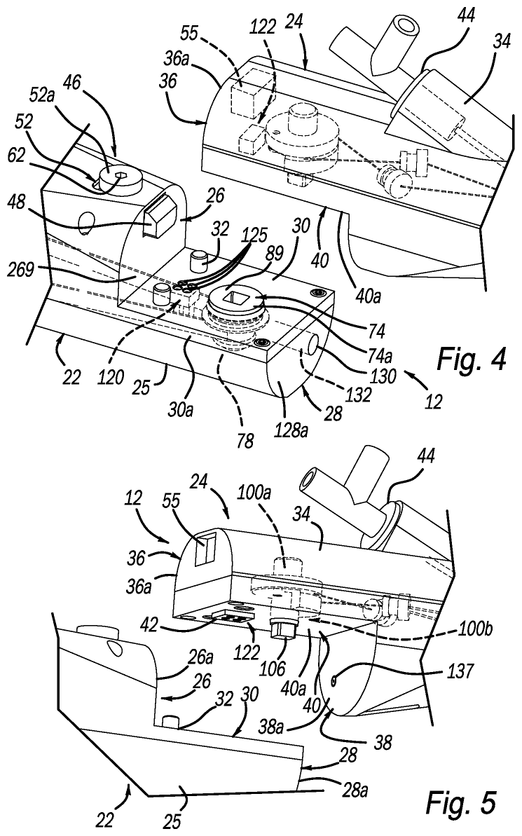

With reference to FIGS. 3-6 and 13, a female electrical coupling 120 is associated with the reusable first body 22 (FIGS. 4 and 6) and a male electrical coupling 122 (FIGS. 3-5 and 13) is associated with the disposable second body 24. The electrical couplings 120, 122 are electrically coupled to each other at a location where the lateral surface 40a of the wall 40 abuts against the lateral surface 30a of the wall 30. The electrical couplings 120, 122 are also electrically coupled to each other at a location between the attachment member 48 and the drive pulley 74. The electrical coupling 120 may be partially disposed within the cavity 70 of the casing 25 of the reusable first body 22. The electrical coupling 122 may be partially disposed within the cavity 98 of the casing 34 of the disposable second body 24 and may include one or more spring pin connectors extending through the wall 40 of the disposable second body 24. The spring pin connectors extend through openings 125 in the wall 30 of the reusable first body 22 to electrically connect to the electrical coupling 120. In this way, electrical devices such as a camera, for example, may be electrically coupled to the endoscope 10.

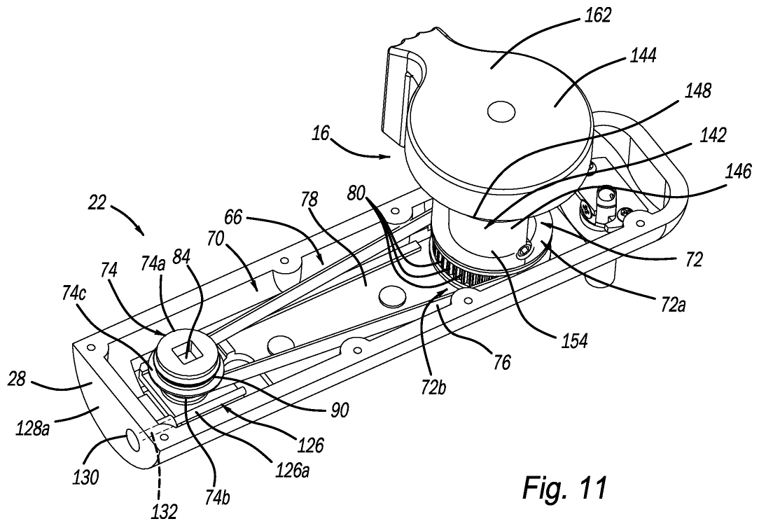

As shown best in FIGS. 3A and 3B and 11, a first fiber bundle 126 is at least partially disposed within the cavity 70 of the casing 25. The first fiber bundle 126 has a first end electrically connected to an illumination source (not shown) and a second end 128a adjacent to a transparent window 130 (FIGS. 3A, 3B, 4, 6, 8, and 11) formed in the wall 28 of the reusable first body 22. A ferrule 132 is fixed to the window 130 and surrounds the second end 128a of the first fiber bundle 126. In some configurations, the ferrule 132 or any other suitable protective covering may surround the entire first fiber bundle 126. The first fiber bundle 126 may be secured to the plate 78 and/or the casing 25. In other configurations, a light emitting diode (LED) may be disposed in the cavity 70 of the casing 25 instead of the first fiber bundle 126. In such configurations, the LED may be positioned adjacent the window 130.

As shown best in FIGS. 12 and 13, a second fiber bundle 134 is at least partially disposed within the cavity 98 of the casing 34 and has a diameter that is smaller than a diameter of the first fiber bundle 126. The second fiber bundle 134 also has a first end 136a and a second end (not shown). The first end 136a is adjacent to an opening 137 (FIG. 5) formed in the wall 38 of the disposable second body 24. That is, the first end 136a does not contact the first fiber bundle 126. The first end 136a is also coaxial with the second end 128a of the first fiber bundle 126 when the reusable first body 22 and the disposable second body 24 are coupled to each other. The second end (not shown) is fixed to a distal end 138 of the insertion tube 18. Light emitted from the illumination source is transmitted from the first fiber bundle 126 to the second fiber bundle 134 where it illuminates from the distal end 138 of the insertion tube 18. In this way, when the distal end 138 is received in a cavity of a human body, for example, the cavity is illuminated. A ferrule 140 is disposed proximate to the opening 137 and surrounds the first end 136a of the second fiber bundle 134. In some configurations, the ferrule 140 or any other suitable protective covering may surround the entire second fiber bundle 134. The second fiber bundle 134 may be secured to the casing 34 of the disposable second body 24.

With reference to FIGS. 6 and 8-11, the handle assembly 16 includes a connecting member 142 and a knob 144. The connecting member 142 is at least partially disposed within the cavity 70 of the casing 25 and includes a cylindrical-shaped body 146 and a flange 148. The body 146 extends through an opening in the casing 25 and is rotationally fixed to the knob pulley 72 so that rotation of the connecting member 142 causes corresponding rotation of the knob pulley 72. A sealing member 150 (e.g., an O-ring) is received in an annular groove formed in an outer cylindrical surface 154 of the body 146. The sealing member 150 is sealingly engaged with an inner cylindrical surface of the opening in the casing 25 so that fluid and debris, for example, are inhibited from entering into the cavity 70.

The flange 148 extends from an end of the body 146 and includes an outer circumferential surface 155 having a diameter that is greater than a diameter of the outer cylindrical surface 154 of the body 146. The flange 148 is also externally located relative to the casing 25 and includes a flat surface 156 having a plurality of first apertures 158 and a central second aperture 160. The first apertures 158 are circumferentially spaced apart around the second aperture 160.

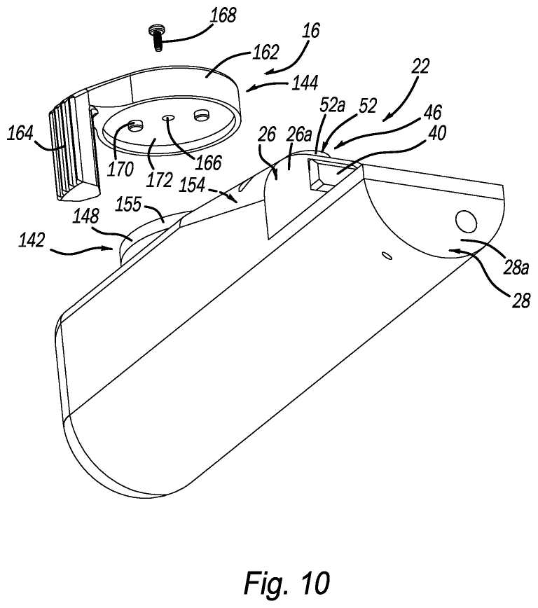

The knob 144 is removably coupled to the flange 148 and includes a body 162 and a plurality of gripping parts 164. The body 162 includes a central aperture 166 extending therethrough. A fastener 168 (FIG. 10) such as a screw or bolt may extend through the apertures 160, 166 to couple the knob 144 and the connecting member 142 to each other. The body 162 also includes a boss 170 extending from a flat surface 172 thereof. The boss 170 is configured to be received in one of the first apertures 158 of the flange 148. In this way, the knob 144 can be positioned in multiple positions based on the preference of the user. Each gripping part 164 is fixed at a periphery of the body 162 and is gripped by a user. That is, when the knob 144 is secured to the connecting member 142 as described above, the user may grip the pair of gripping parts 164 and rotate the knob 144. Rotation of the knob 144 rotates the connecting member 142 and the knob pulley 72, which, in turn, rotates the drive pulley 74 via the transmission element 76. This causes the driven pulley 100 to rotate, which causes the wires 102a, 102b to articulate the bending section 20.

The handle assembly 16 of the present disclosure provides the benefit of allowing the knob 144 to be sterilized separately from the connecting member 142 which is coupled to the reusable first body 22. The knob 144 of the handle assembly is also usable by both left-handed and right-handed users.

The insertion tube 18 is configured to be received in the cavity of the human body, for example, and includes a proximal end 174 and the distal end 138. The proximal end 174 is coupled to the disposable second body 24 and the distal end 138 is coupled to the bending section 20.

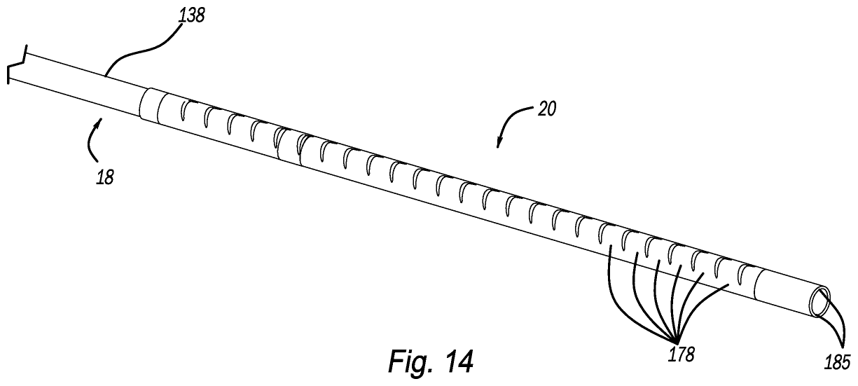

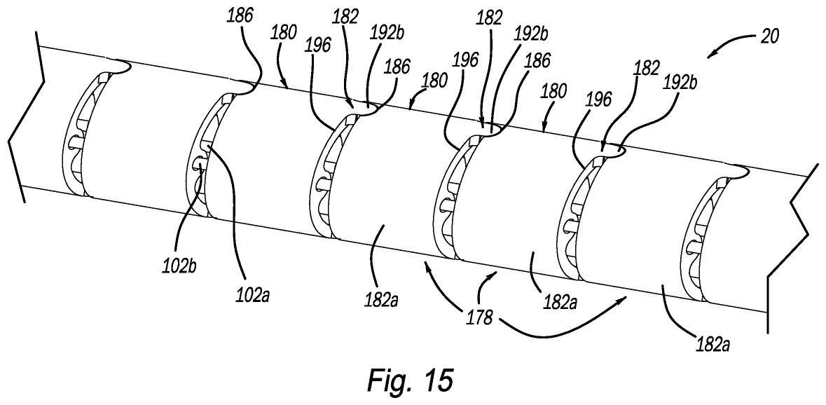

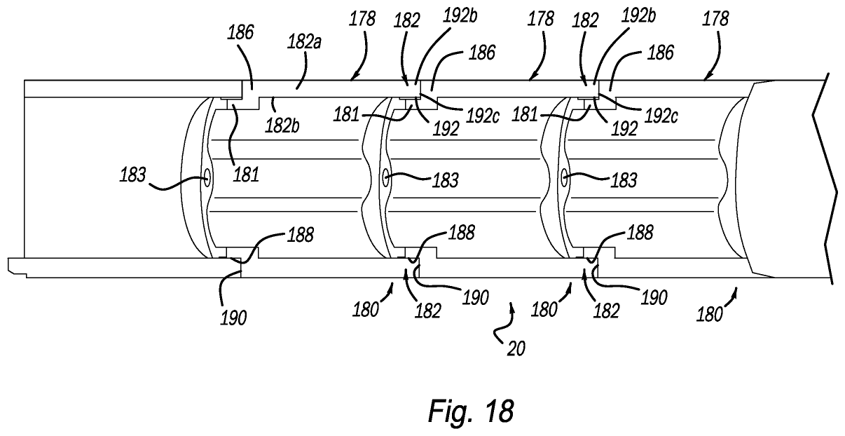

With reference to FIGS. 14-18, the bending section 20 includes a series of stacked links 178 movably coupled to each other. Each link 178 is generally cylindrical shaped and includes a body 180, a pair of reinforcement walls 181, and a pair of protrusions 182 (the outer most link 178 does not include the pair of protrusions). The body 180 includes an outer cylindrical surface 182a and an inner cylindrical surface 182b. The body 180 also defines a pair of channels 183 and a pair of recesses 184. The pair of channels 183 are diametrically opposed to each other and extend through the body 180. Each wire 102a, 102b of the transmission element 102 extends through a respective channel 183 of the body 180, thereby providing tension and holding the links 178 together. The wires 102a, 102b are also configured to articulate the series of stacked links 178. A cylindrically shaped end portion 185 is secured to the second end of each wire 102a, 102b. The end portion 185 has a diameter that is greater than a diameter of the channels 183 to inhibit the wires 102a, 102b from disengaging from the links 178 and the links 178 from disengaging from each other.

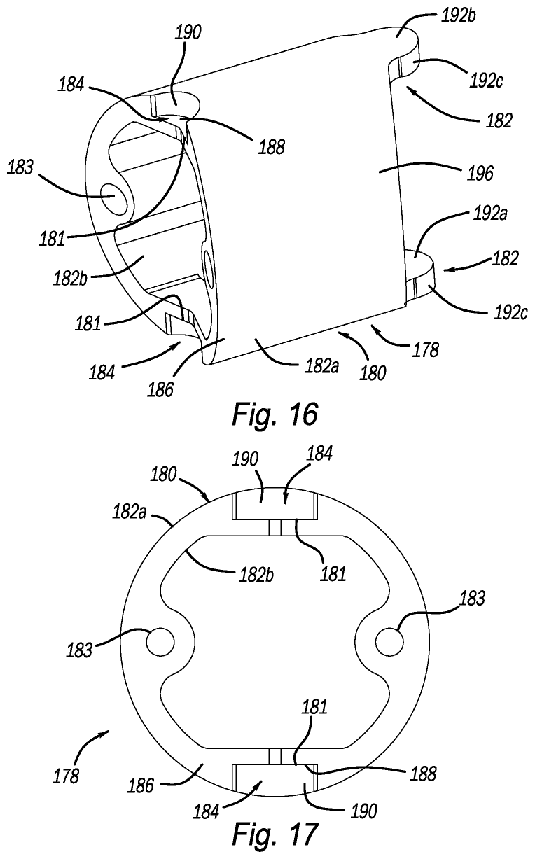

As shown in FIGS. 16 and 17, the pair of recesses 184 are diametrically opposed to each other. Each recess 184 is formed in the body 180 at a first axial end 186 and includes an arcuate surface 190. The pair of reinforcement walls 181 are fixed to the body 180 proximate the pair of recesses 184. Each reinforcement wall 181 includes an engagement surface 188 that faces away from the inner cylindrical surface 182b and in the same direction as the outer cylindrical surface 182a.

The pair of protrusions 182 extend from a second axial end 196 of the body 180 in an axial direction. The pair of protrusions 182 are also diametrically opposed to each other and includes an inner surface 192a, an outer surface 192b, and an arcuate end surface 192c. The inner surface 192a faces away from the outer surface 192b. Each protrusion 182 is configured to be received in a respective recess 184 such that the inner surface 192a of each protrusion 182 abuts against the engagement surface 188 of a respective reinforcement wall 181 and the arcuate end surface 192c of each protrusion 182 abuts against the arcuate surface 190 of the respective recess 184. The bending section 20 of the present disclosure provides the benefit of inhibiting a twisting motion when the transmission element 102 articulates the bending section 20. The bending section 20 of the present disclosure also provides the benefit of coupling the links 178 to each other without the use of fasteners such as rivets, for example.

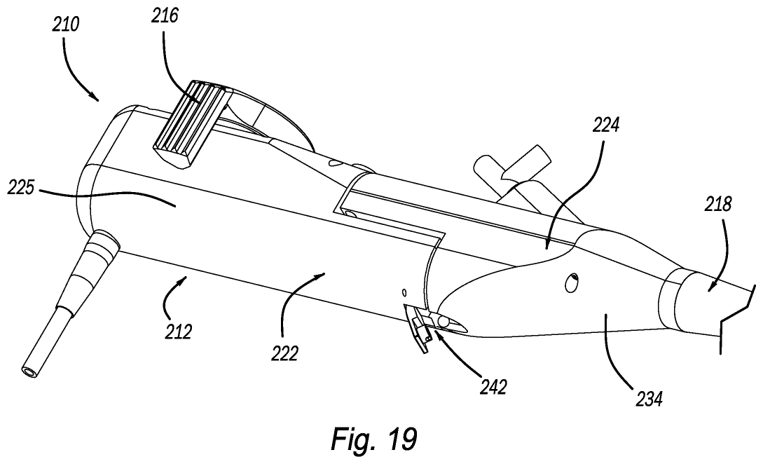

With reference to FIGS. 19-21, another endoscope 210 is illustrated. The structure and function of the endoscope 210 is similar or identical to that of the endoscope 10 described above, apart from any exceptions noted below.

The endoscope 210 includes a control body 212, an angulation drive assembly (not shown), a handle assembly 216, a flexible insertion tube 218, and a bending section or articulation mechanism (not shown). The control body 212 is configured to be grasped or held by a user and may be a generally cylindrical shape as shown. The control body 212 is similar to the control body 12 described above, apart from any exceptions noted below. The control body 212 includes a reusable first body 222 and a disposable second body 224. The reusable first body 222 includes a casing 225 and a plurality of walls (not shown). The casing 225 includes a recess 240 formed adjacent a transversely extending wall (not shown) of the plurality of walls of the first body 222. The disposable second body 224 is removably coupled to the reusable first body 222 and includes a casing 234 and a plurality of walls (not shown). The casing 234 includes a recess 241 formed adjacent a transversely extending wall (not shown) of the plurality of walls of the second body 224. The recess 241 of the casing 234 is aligned with the recess 240 of the casing 225.

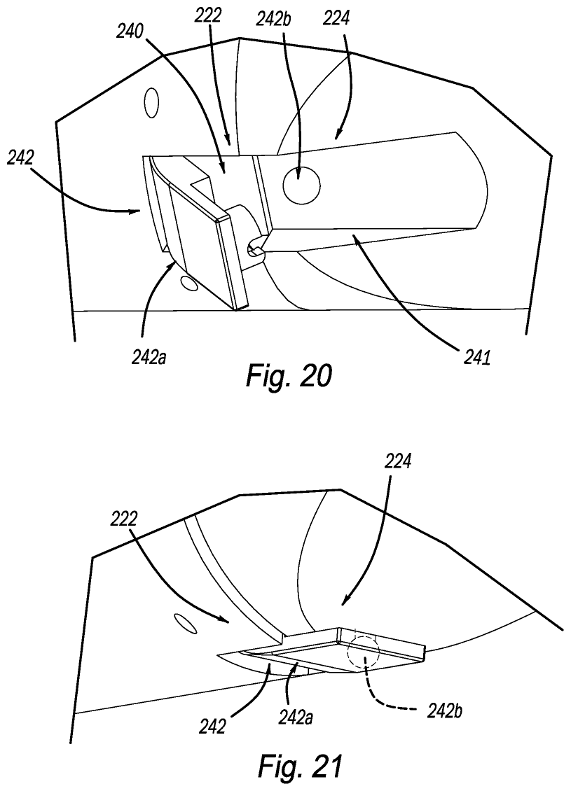

An attachment mechanism 242 is associated with the control body 212 and is configured to further secure the first body 222 and the second body 224 to each other, thereby inhibiting sliding and torsional movements, for example, of the first and second bodies 222, 224 relative to each other. The attachment mechanism 242 is associated with a surface of the control body 212 that is diametrically opposite a surface of the control body 212 including a coupling assembly (i.e., an assembly coupling the first and second bodies 222, 224 to each other). The attachment mechanism 242 includes a first attachment member or feature 242a and a second attachment member or feature 242b. In the example illustrated, the first attachment member 242a is rotatably coupled to the casing 225 between a secured position (FIG. 21) in which the first attachment member 242a is received in the recesses 240, 241 and a release position (FIG. 20) in which the first attachment member 242a is removed from the recess 241 and at least partially removed from the recess 240. When in the secured position, the first attachment member 242a and the second attachment member 242b form a snap fit. In the example illustrated, the second attachment member 242b is a protrusion fixed in the recess 241 of the casing 234 of the second body 224. In the example illustrated, the protrusion has a spherical shape, however, it is understood that the protrusion can include other suitable shapes such as a cylindrical shape, for example. In some forms, the first attachment member 242a can be rotatably coupled to the casing 234 of the second body 224 and the second attachment member 242b can be fixed in the recess 240 of the casing 225 of the first body 222.

The angulation drive assembly, the handle assembly 216, the flexible insertion tube 218, and the bending section are similar or identical to the angulation drive assembly 14, the handle assembly 16, the flexible insertion tube 18, and the bending section 20, respectively, described above, and therefore, will not be described again in detail.

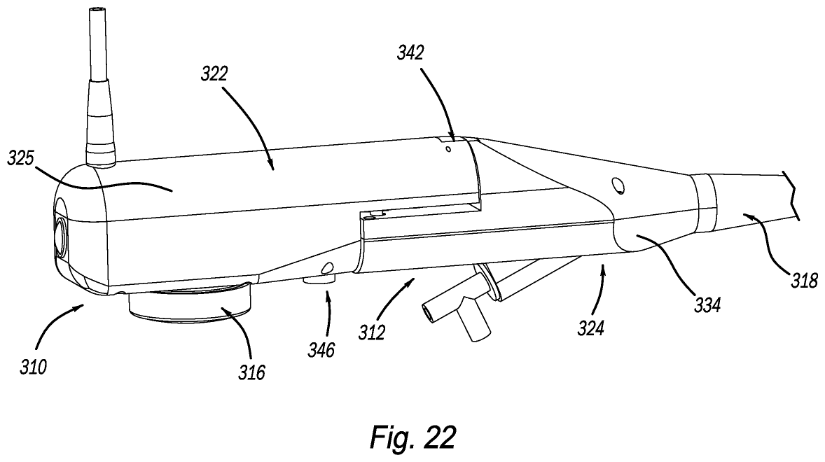

With reference to FIGS. 22-24, another endoscope 310 is illustrated. The structure and function of the endoscope 310 is similar or identical to that of the endoscope 10 described above, apart from any exceptions noted below.

The endoscope 310 includes a control body 312, an angulation drive assembly (not shown), a handle assembly 316, a flexible insertion tube 318, and a bending section or articulation mechanism (not shown). The control body 312 is configured to be grasped or held by a user and may be a generally cylindrical shape as shown. The control body 312 is similar to the control body 12 described above, apart from any exceptions noted below. The control body 312 includes a reusable first body 322 and a disposable second body 324. The reusable first body 322 includes a casing 325 and a plurality of walls (not shown). The disposable second body 324 is removably coupled to the reusable first body 322 and includes a casing 334 and at least one transversely extending wall 341 (FIG. 24).

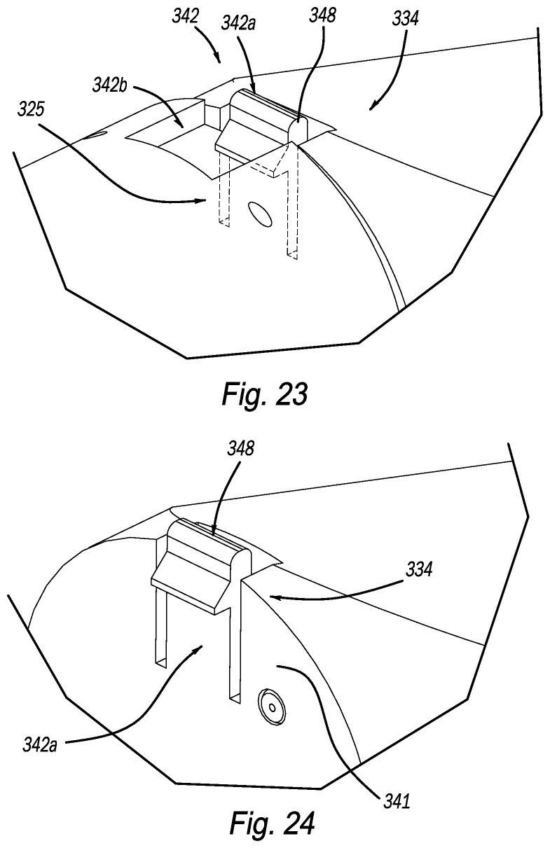

An attachment mechanism 342 is associated with the control body 312 and is configured to further secure the first body 322 and the second body 324 to each other, thereby inhibiting sliding and torsional movements, for example, of the first and second bodies 322, 324 relative to each other. The attachment mechanism 342 includes a first attachment member or feature 342a and a second attachment member or feature 342b (FIG. 23). In the example illustrated, the first attachment member 342a is a flexible tab having a barbed tip 348 and the second attachment member 342b is a recess. The first attachment member 342a is formed in the wall 341 of the second body 324 and snaps into engagement with the casing 325 of the first body 322 when the first and second bodies 322, 324 are secured to each other. Stated differently, the barbed tip 348 of the flexible tab snaps into engagement with the recess, which is formed in the casing 325 adjacent to a transversely extending wall (not shown) of the plurality of walls of the first body 322. The attachment mechanism 342 secures the first and second bodies 322, 324 at a location that is diametrically opposite a surface of the control body 312 including coupling assembly 346 (FIG. 22; an assembly coupling the first and second bodies 322, 324 to each other). In some forms, the first attachment member 342a (i.e., flexible tab) can be secured to the first body 222 and the second attachment member 342b (i.e., recess) can be formed in the second body 224.

The angulation drive assembly, the handle assembly 316, the flexible insertion tube 318, and the bending section are similar or identical to the angulation drive assembly 14, the handle assembly 16, the flexible insertion tube 18, and the bending section 20, respectively, described above, and therefore, will not be described again in detail.

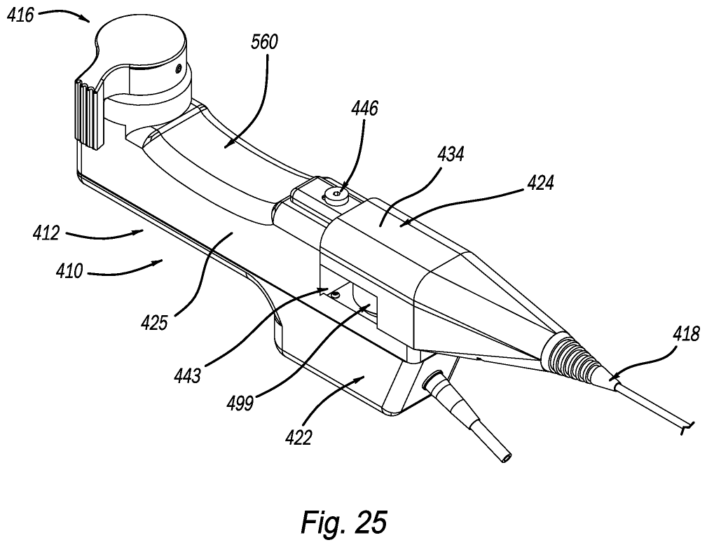

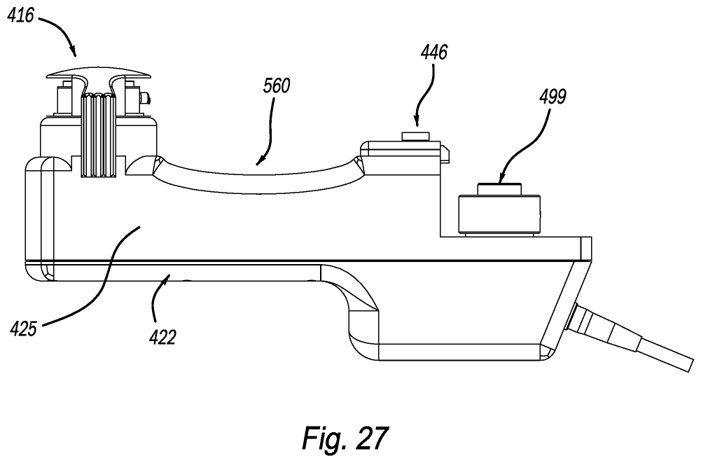

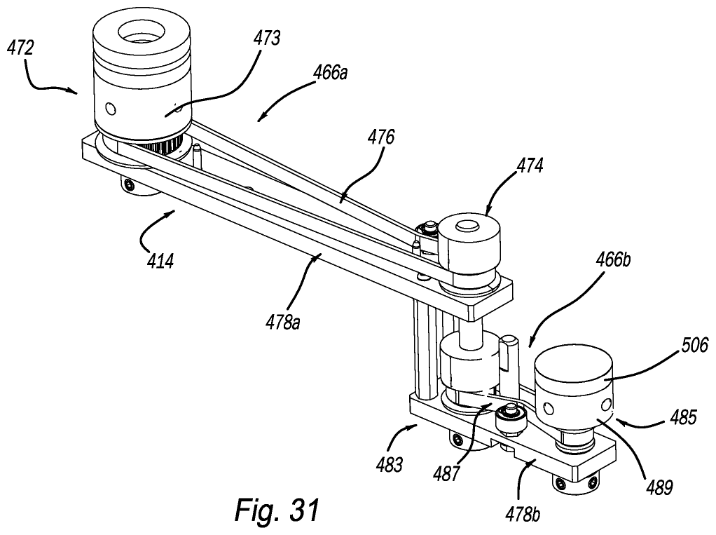

With reference to FIGS. 25-32, another endoscope 410 is illustrated. The structure and function of the endoscope 410 is similar or identical to that of the endoscope 10 described above, apart from any exceptions noted below.

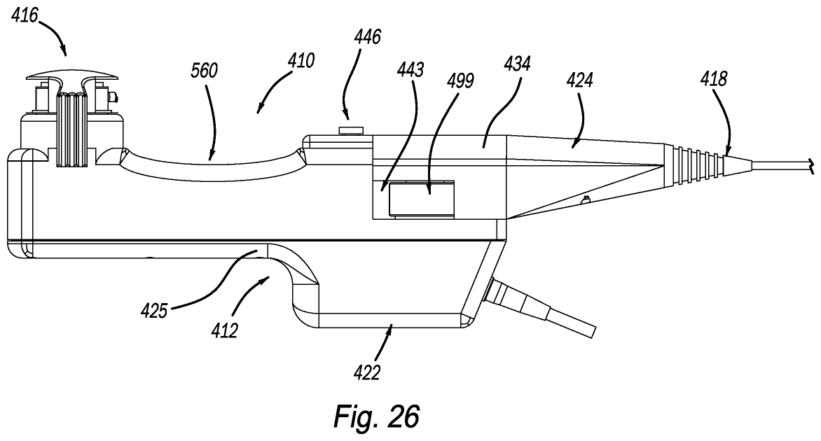

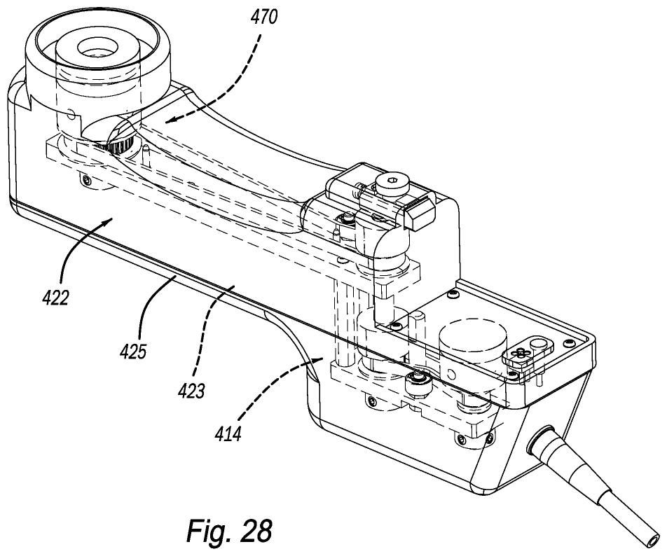

The endoscope 410 includes a control body 412 (FIGS. 25 and 26), an angulation drive assembly 414 (FIGS. 28, 31, and 32), a handle assembly 416 (FIGS. 25-27, 29, and 32), a flexible insertion tube 418 (FIGS. 25, 26, and 30), and a bending section or articulation mechanism (not shown). The control body 412 is configured to be grasped or held by a user. The control body 412 includes a reusable first body 422 and a disposable second body 424. The reusable first body 422 includes a casing 425, a first upper wall 426, a second upper wall 428 and an intermediate wall 430. In the example illustrated, the casing 425 comprises an upper shell and a lower shell that are mechanically fastened to each other. In some forms, the casing 425 is made of a single, monolithic structure as opposed to two shells mechanically fastened to each other. A sealing member 423 (FIG. 28) is disposed within an internal groove of the casing 425 and extends around an internal periphery to inhibit debris and fluid from entering into a cavity 470 (FIG. 28) of the casing 425. The first upper wall 426 is fixed to the casing 425 and has a generally semi-circular shape. The first upper wall 426 extends in a transverse direction of the endoscope 410 and includes a flat upper surface. Similarly, the second upper wall 428 is fixed to the casing 425 and extends in a transverse direction of the endoscope 410. The second upper wall 428 is also inclined from a first end furthest away from the intermediate wall 430 toward an opposing second end adjacent or near the intermediate wall 430. The intermediate wall 430 is removably coupled to the casing 425 via fasteners, for example, and extends in a longitudinal direction of the endoscope 410. The walls 426, 428, 430 cooperate with each other to form a stepped interface.

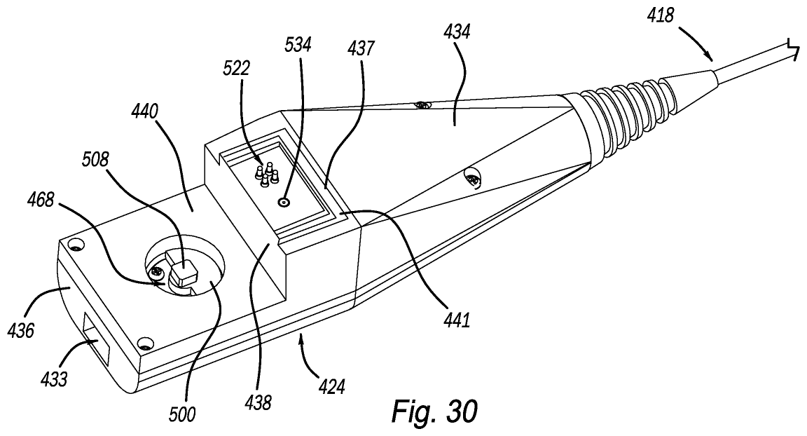

With reference to FIG. 30, the disposable second body 424 is removably coupled to the reusable first body 422 and includes a casing 434, a first lower wall 436, a second lower wall 438, an intermediate wall 440, and an outer wall 441. The first lower wall 436 is fixed to the casing 434 and has semi-circular shape. The first lower wall 436 extends in a transverse direction of the endoscope 410 and includes a flat lower surface. The first lower wall 436 also includes a groove 433 configured to receive a portion of the coupling assembly 446. The second lower wall 438 is fixed to the casing 434 and has a generally rectangular shape. The second lower wall 438 also extends in a transverse direction of the endoscope 410 and includes a flat lower surface. The intermediate wall 440 is removably coupled to the casing 434 via fasteners, for example, and extends in a longitudinal direction of the endoscope 410. The outer wall 441 extends parallel to the intermediate wall 440 and is vertically offset from the intermediate wall 440. The outer wall 441 also extends perpendicular to the first and second lower walls 436, 438 and includes a protrusion 437 extending therefrom. The walls 436, 438, 440, 441 cooperate with each other to form a stepped interface.

When the reusable first body 422 and the disposable second body 424 are connected to each other, the first lower wall 436 of the second body 424 abuts against a portion of the first upper wall 426 of the first body 422 and the protrusion 437 of the outer wall 441 is received in a recess 439 of the intermediate wall 430 so that the outer wall 441 of second body 424 abuts against a portion of the intermediate wall 430 of the first body 422. In this way, the intermediate wall 430 of the first body 422 is spaced apart and opposite from the intermediate wall 440 of the second body 422 and the upper wall 426 of the first body 422 is spaced apart and opposite from the second lower wall 438 of the second body 424, thereby forming a compartment 443 between the first and second bodies 422, 424. The protrusion 437 of the outer wall 441 being received in the recess 439 of the intermediate wall 430 also inhibits longitudinal and lateral movement of the first and second bodies 422, 424 relative to each other.

The coupling assembly 446 is associated with the reusable first body 422 and is configured to secure the first and second bodies 422, 424 to each other. The structure and function of the coupling assembly 446 is similar or identical to the coupling assembly 46 described above, and therefore, will not be described again in detail.

With reference to FIGS. 28, 31 and 32, the angulation drive assembly 414 is coupled to the bending section and is operable to articulate the bending section. The angulation drive assembly 414 includes first pulley devices 466a, 466b and a second pulley device 468 operatively connected to the pulley device 466b. The first pulley devices 466a, 466b are associated with the reusable first body 422. For example, the first pulley devices 466a, 466b are entirely disposed within the cavity 470 of the reusable first body 422. The first pulley device 466a includes a knob pulley 472, a drive pulley 474, and a transmission element 476. The knob pulley 472 is rotationally fixed to the handle assembly 416. In some configurations, as shown in the figures, the knob pulley 472 is rotatably engaged to a plate 478a that is disposed within and secured to the casing 425 of the reusable first body 422. In one example, the plate 478a may be secured to the casing 425 via fasteners such as screws, bolt, and rivets, among others. In another example, the plate 478a may be secured to locking features (e.g., grooves) formed in the casing 425 of the reusable first body 422.

The knob pulley 472 is generally cylindrical shape and includes a handle portion 472a, a transmission element portion 472b, and an end portion 472c. The handle portion 472a is rotationally fixed to the handle assembly 416 such that rotation of the handle assembly 416 causes corresponding rotation of the knob pulley 472. The handle portion 472a comprises a permanent magnet 473 such as a rare earth magnet, for example. The transmission element portion 472b extends from the handle portion 472a and includes a plurality of teeth formed around an outer circumferential surface thereof. The transmission element portion 472b is positioned between the handle portion 472a and the end portion 472c. The end portion 472c includes an inner section 475a and an outer section 475b. The inner section 475a extends at least partially through the plate 478a such that the plate 478a rotatably supports the inner section 475a. The outer section 475b of the end portion 472c and a flange portion 472d positioned between the inner section 475a and the transmission element portion 472b include diameters that are greater than a diameter of the inner section 475a. In this way, a rotational axis of the knob pulley 472 is rotationally fixed (i.e., the knob pulley 472 is inhibited from moving laterally, vertically, or longitudinally).

The drive pulley 474 is operatively connected to the knob pulley 472 and rotationally fixed to the pulley device 466b so that rotation of the drive pulley 474 operates the pulley device 466b. The drive pulley 474 is generally cylindrical shape and includes a transmission element portion 474a, a plate portion 474b, a connecting portion 474c, and a flange portion 474d. The transmission element portion 474a includes a plurality of teeth formed around an outer circumferential surface thereof. The plate portion 474b extends at least partially through the plate 478a such that the plate 478a rotatably supports the plate portion 474b. The plate portion 474b is positioned between the transmission element portion 474a and the flange portion 474d. The transmission element portion 474a and the flange portion 474d include diameters that are greater than a diameter of the plate portion 474b. In this way, a rotational axis of the drive pulley 474 is rotationally fixed (i.e., the drive pulley 474 is inhibited from moving laterally, vertically, or longitudinally).

The transmission element 476 may be a timing belt, for example, and may include teeth (not shown) on an inner surface thereof. The teeth of the transmission element 476 are meshingly engaged with teeth of the knob pulley 472 and with teeth of the drive pulley 474. In this way, rotation of the knob pulley 472 via the handle assembly 416 is transmitted to the drive pulley 474 via the transmission element 476, thereby causing corresponding rotation of the drive pulley 474. One or more guide members 477 are secured to the plate 478a adjacent the transmission element 476 and are configured to guide the transmission element 476 as the transmission element 476 transmits rotational motion of the knob pulley 472 to the drive pully 474.

The pulley device 466b is operatively connected to the pulley device 466a and the second pulley device 468 such that rotational force generated by the pulley device 466a is transmitted to the second pulley device 468 via the pulley device 466b. The pulley device 466b comprises a driven pulley 483, a coupler pulley 485 and a transmission element 487. The driven pulley 483 is generally cylindrical shape and includes an attachment portion 483a, a transmission element portion 483b, a flange portion 483c and an end portion 483d. The attachment portion 483a is rotationally fixed to the connection portion 474c of the drive pulley 474 such that rotation of the drive pulley 474 causes corresponding rotation of the driven pulley 483. For example, the attachment portion 483a includes a locking groove (not shown) comprising a shape that corresponds to the shape of the connection portion 474c. The connecting portion 474c extends into the locking groove of the attachment portion 483a, thereby rotationally fixing the drive pulley 474 and the driven pulley 483 to each other.

The transmission element portion 483b extends from the attachment portion 483a and includes a plurality of teeth formed around an outer circumferential surface thereof. The transmission element portion 483b is positioned between the attachment portion 483a and the flange portion 483c. The end portion 483d includes an inner section 484a and an outer section 484b. The inner section 484a extends at least partially through a plate 478b such that the plate 478b rotatably supports the inner section 484a. The outer section 484b of the end portion 483d is positioned on a side of the plate 478b opposite the flange portion 483c, and the outer section 484b of the end portion 483d and the flange portion 483c include diameters that are greater than a diameter of the inner section 484a. In this way, a rotational axis of the driven pulley 483 is rotationally fixed (i.e., the driven pulley 483 is inhibited from moving laterally, vertically, or longitudinally). One or more connecting rods 486 connect the plates 478a, 478b to each other within the cavity 470 of the first body 422.

The coupler pulley 485 is operatively connected to the driven pulley 483 and rotationally fixed to the second pulley device 468 so that rotation of the driven pulley 483 operates the second pulley device 468. The coupler pulley 485 is generally cylindrical shape and includes a coupler portion 485a, a transmission element portion 485b, a flange portion 485c, and an end portion 485d. The coupler portion 485a comprises a permanent magnet 489 such as a rare earth magnet, for example. The transmission element portion 485b includes a plurality of teeth formed around an outer circumferential surface thereof. The flange portion 485c is positioned between the transmission element portion 485b and the end portion 485d. The end portion 485d includes an inner section 496a and an outer section 496b. The inner section 496a extends at least partially through the plate 478b such that the plate 478b rotatably supports the inner section 496a. The outer section 496b of the end portion 485d is positioned on a side of the plate 478b opposite the flange portion 485c, and the outer section 496b and the flange portion 485c include diameters that are greater than a diameter of the inner section 496a. In this way, a rotational axis of the coupler pulley 485 is rotationally fixed (i.e., the coupler pulley 485 is inhibited from moving laterally, vertically, or longitudinally).

The transmission element 487 may be a timing belt, for example, and may include teeth (not shown) on an inner surface thereof. The teeth of the transmission element 487 are meshingly engaged with teeth of the driven pulley 483 and with teeth of the coupler pulley 485. In this way, rotation of the driven pulley 483 is transmitted to the coupler pulley 485 via the transmission element 487, thereby causing corresponding rotation of the coupler pulley 485. One or more guide members 492 are secured to the plate 478b adjacent the transmission element 487 and are configured to guide the transmission element 487 as the transmission element 487 transmits rotational motion of the driven pulley 483 to the coupler pully 485. One or more connecting rods 498 connect the plate 478b to the first body 422.

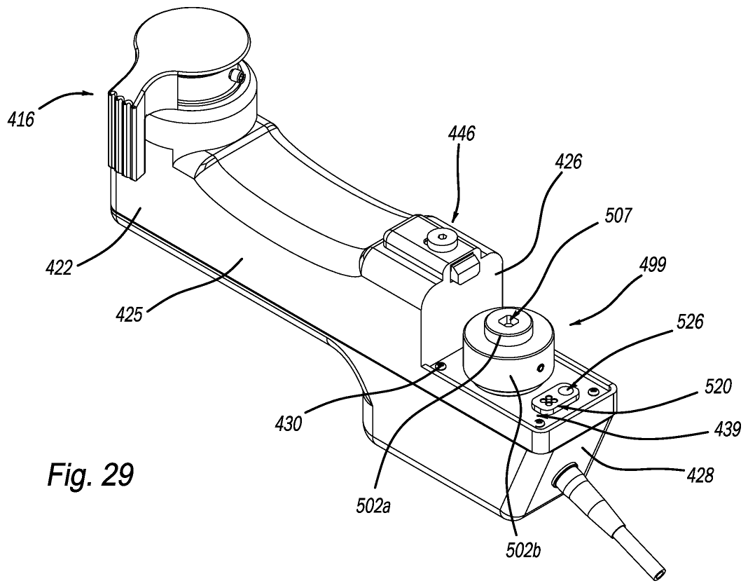

The second pulley device 468 is associated with the disposable second body 424. For example, the second pulley device 468 is at least partially disposed within a cavity of the casing 434 and includes a coupling mechanism 499, a connecting pulley 500 and a transmission element (not shown). The coupling mechanism 499 is disposed within the compartment 443 formed by the first and second bodies 422, 424 and is rotationally secured to the connecting pulley 500 and the coupler pulley 485. The coupling mechanism 499 is located external relative to the cavity 470 of the first body 422 and includes a coupler housing 502, a magnet housing 504 and a permanent magnet 506. The coupler housing 502 is generally cylindrical shape and includes a coupling portion 502a and a housing portion 502b. The coupling portion 502a extends in an axial direction from the housing portion 502b into an opening in the intermediate wall 440 of the second body 424. The coupling portion 502a includes a locking groove 507 (FIG. 29) configured to receive a portion 508 (FIG. 30) of the connecting pulley 500. The locking groove 507 comprises a shape the corresponds to a shape of the portion 508 of the connecting pulley 500 so that, when the portion 508 is inserted into the locking groove 507, the coupling mechanism 499 is rotationally fixed to the connecting pulley 500.

The magnet housing 504 is at least partially housed in the housing portion 502a of the coupler housing 502 and abuts against the intermediate wall 430 of the first body 422. The permanent magnet 506 is at least partially housed in the magnet housing 504 and is attracted to the permanent magnet 489 of the coupler pulley 485. That is, the permanent magnet 506 produces a magnetic force and the permanent magnet 489 produces a magnetic force that are attracted to each other. In this way, the coupler pulley 485 is rotationally fixed to the connecting pulley 500 and the first and second bodies 422, 424 are magnetically coupled to each other. In the example illustrated, fasteners extend through the coupler housing 502, the magnet housing 504, and the permanent magnet 506, thereby securing the coupler housing 502, the magnet housing 504, and the permanent magnet 506 to each other. In some forms, the coupler housing 502 is force fitted onto the magnet housing 504 and the magnet housing 504 is force fitted onto the permanent magnet 506. The structure and function of the connecting pulley 500 is similar or identical to the driven pulley 100 described above, and therefore, will not be described again in detail.

With reference to FIGS. 29 and 30, a female electrical coupling 520 is associated with the reusable first body 422 (FIG. 29; e.g., disposed within the recess 439 of the intermediate wall 430) and a male electrical coupling 522 is associated with the disposable second body 524 (FIG. 30; e.g., extending from the protrusion 437 of the outer wall 441). The electrical couplings 520, 522 are electrically coupled to each other at a location where the outer wall 441 of the second body 424 abuts against the intermediate wall 430 of the first body 422. The structure and function of the female electrical coupling 520 and the male electrical coupling are similar or identical to the female electrical coupling 120 and the male electrical coupling 122, respectively, described above, and therefore, will not be described again in detail below.

A first fiber bundle 526 is at least partially disposed within the cavity 470 of the casing 425. The first fiber bundle 526 has a first end electrically connected to an illumination source (not shown) and a second end adjacent to a transparent window formed in the intermediate wall 430 of the reusable first body 22. A second fiber bundle 534 is at least partially disposed within the casing 434 of the second body 424. The second fiber bundle 534 has a first end adjacent to an opening formed in the protrusion 437 of the disposable second body 424 and a second end is fixed to a distal end of the insertion tube 418. Light emitted from the illumination source is transmitted from the first fiber bundle 526 to the second fiber bundle 534 where it illuminates from the distal end of the insertion tube 418. In this way, when the distal end is received in a cavity of a human body, for example, the cavity is illuminated

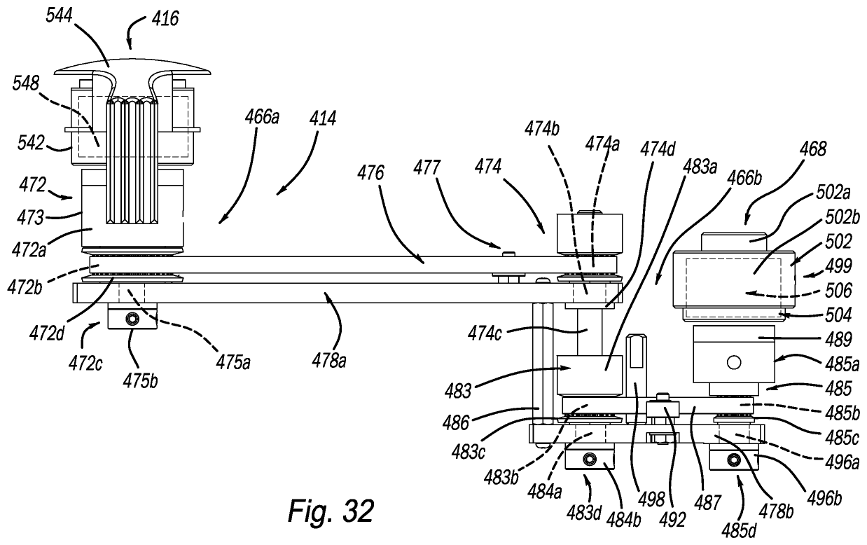

With reference to FIG. 32, the handle assembly 416 includes a connecting member 542 and a knob 544. The connecting member 542 is located external relative to the cavity 470 of the first body 422 and is rotationally fixed to the knob pulley 472 such that rotation of the knob 544 causes corresponding rotation of the knob pulley 472. The connecting member 542 has a generally cylindrical shape and includes a permanent magnet 548. In the example illustrated, the connecting member 542 is partially disposed within a circular groove formed in the casing 425 of the first body 422 to inhibit movement of the connecting member 542 in the lateral and longitudinal direction. The permanent magnet 548 is attracted to the permanent magnet 473 of the knob pulley 472. That is, the permanent magnet 548 produces a magnetic force and the permanent magnet 473 produces a magnetic force that are attracted to each other. In this way, the knob pulley 472 is rotationally fixed to the handle assembly 416.

The knob 544 is rotationally fixed to the connecting member 542 and the permanent magnet 548. In the example illustrated, one or more fasteners extend through the knob 544, the connecting member 542, and the permanent magnet 548, thereby rotationally fixing the knob 544, the connecting member 542, and the permanent magnet 548 to each other. In some forms, the knob 544 is force fitted onto the connecting member 542 and the connecting member 542 is force fitted onto the permanent magnet 548.

The structure and function of the knob 544 is similar or identical to knob 44 described above, and therefore, will not be described again in detail. Rotation of the knob 544 rotates the connecting member 542 and the knob pulley 472, which, in turn, rotates the drive pulley 474 via the transmission element 476. This causes the driven pulley 483, the coupler pulley 485 and the connecting pulley 500 to rotate, which causes the wires, for example, to articulate the bending section. A portion 560 of the casing 425 between the handle assembly 416 and the coupling assembly 446 has a concave shape (comprises a surface that curves inwardly) to facilitate the grip of the control body 412 by the user. To detach the first and second bodies 422, 424 from each other, the user first moves the coupling assembly 446 to the unlocked position. Then, the user pulls the first and second bodies 422, 424 away from each other such that a pulling force exerted by the user is greater than the magnetic force attracting the magnets 489, 506 to each other and the magnets 473, 548 to each other.

The structure and function of flexible insertion tube 418 is similar or identical to the flexible insertion tube 18 described above, and therefore, will not be described again in detail. The structure and function of the bending section is similar or identical to the bending section 20 described above, and therefore, will not be described again in detail.

The endoscope 410 of the present disclosure rotationally fixes the handle assembly 416 and the second pulley device 468 to the first pulley devices 466a, 466b of the first body 422 using a magnetic force. In this way, improved sealing of the cavity 470 of the first body 422 is achieved by reducing the number of openings or apertures formed in the first body 422.

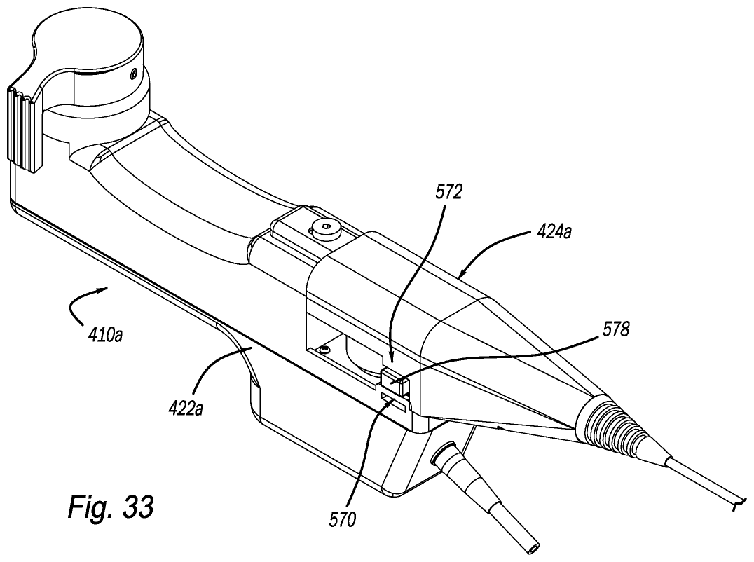

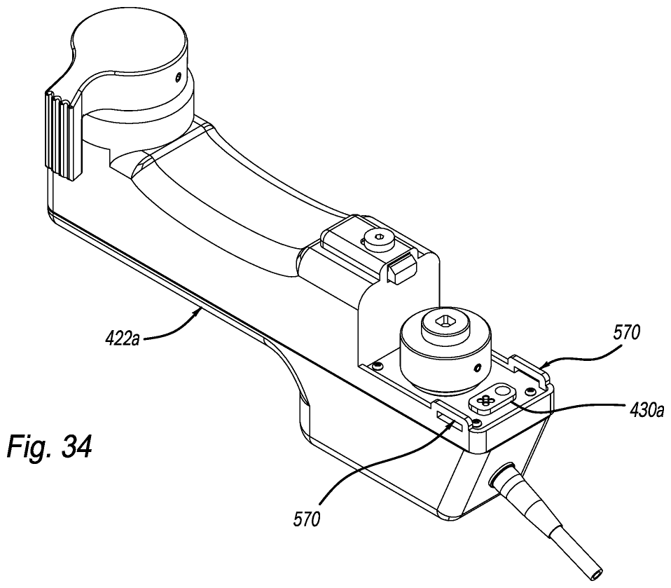

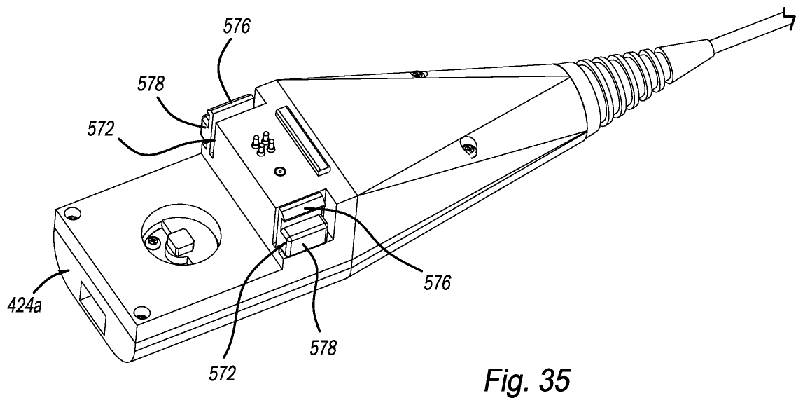

In some forms, as shown in FIGS. 33-35, the endoscope 410a includes a first body 422a comprising a pair of connecting members 570 and a second body 424a comprising a pair of flexible tabs 572. The connecting members 570 are secured to opposites sides of an intermediate wall 430a of the first body 422a at or near a periphery. Each flexible tab 572 of the pair of flexible tabs 572 is cantilevered to an opposite side of the second body 424a and includes a barbed tip 576 and a release member 578. The flexible tabs 572 are moveable between a first position in which the barbed tips 576 snap into engagement with openings formed in the connecting members 570, thereby further securing the first and second bodies 422a, 424a to each other, and a second position in which the flexible tabs 572 are removed from the openings to allow detachment of the first and second bodies 422a, 422b from each other. To move the flexible tabs 572 from the first position to the second position, a user presses the release members 578 so that the flexible tabs 572 deflect inwardly causing the barbed tips 576 to be removed from the openings of the connecting members 570.

Unless otherwise expressly indicated herein, all numerical values indicating mechanical/thermal properties, compositional percentages, dimensions and/or tolerances, or other characteristics are to be understood as modified by the word “about” or “approximately” in describing the scope of the present disclosure. This modification is desired for various reasons including industrial practice, material, manufacturing, and assembly tolerances, and testing capability.

As used herein, the phrase at least one of A, B, and C should be construed to mean a logical (A OR B OR C), using a non-exclusive logical OR, and should not be construed to mean “at least one of A, at least one of B, and at least one of C.”

In this application, the term “controller” and/or “module” may refer to, be part of, or include: an Application Specific Integrated Circuit (ASIC); a digital, analog, or mixed analog/digital discrete circuit; a digital, analog, or mixed analog/digital integrated circuit; a combinational logic circuit; a field programmable gate array (FPGA); a processor circuit (shared, dedicated, or group) that executes code; a memory circuit (shared, dedicated, or group) that stores code executed by the processor circuit; other suitable hardware components (e.g., op amp circuit integrator as part of the heat flux data module) that provide the described functionality; or a combination of some or all of the above, such as in a system-on-chip.

The term memory is a subset of the term computer-readable medium. The term computer-readable medium, as used herein, does not encompass transitory electrical or electromagnetic signals propagating through a medium (such as on a carrier wave); the term computer-readable medium may therefore be considered tangible and non-transitory. Non-limiting examples of a non-transitory, tangible computer-readable medium are nonvolatile memory circuits (such as a flash memory circuit, an erasable programmable read-only memory circuit, or a mask read-only circuit), volatile memory circuits (such as a static random access memory circuit or a dynamic random access memory circuit), magnetic storage media (such as an analog or digital magnetic tape or a hard disk drive), and optical storage media (such as a CD, a DVD, or a Blu-ray Disc).

The apparatuses and methods described in this application may be partially or fully implemented by a special purpose computer created by configuring a general-purpose computer to execute one or more particular functions embodied in computer programs. The functional blocks, flowchart components, and other elements described above serve as software specifications, which can be translated into the computer programs by the routine work of a skilled technician or programmer.

The description of the disclosure is merely exemplary in nature and, thus, variations that do not depart from the substance of the disclosure are intended to be within the scope of the disclosure. Such variations are not to be regarded as a departure from the spirit and scope of the disclosure.

What is claimed is:

1. An endoscope comprising:
a control body including:
a reusable first body including a first stepped interface, the first stepped interface having a first upper surface, a second upper surface, and a first intermediate surface positioned between the first upper surface and the second upper surface;
a disposable second body removably coupled to the first body and including a second stepped interface, the second stepped interface having a first lower surface, a second lower surface, and a second intermediate surface positioned between the first lower surface and the second lower surface;
an insertion tube coupled to the control body;
a bending section secured to a distal end of the insertion tube; and
an angulation drive assembly coupled to the bending section and operable to articulate the bending section,
wherein when the reusable first body and the disposable second body are connected to each other, the first lower surface abuts against the first upper surface, the second lower surface abuts against the second upper surface, and the first intermediate surface abuts against the second intermediate surface.

2. The endoscope according to claim 1, wherein the first intermediate surface and the second intermediate surface extend in a longitudinal direction of the endoscope.

3. The endoscope according to claim 1, wherein the first and second intermediate surfaces are flat.

4. The endoscope according to claim 1, wherein the first and second upper surfaces have a semi-circular shape, and wherein the first and second lower surfaces have a semi-circular shape.

5. The endoscope according to claim 1, wherein the first and second upper surfaces extend orthogonal to the first intermediate surface, and wherein the first and second lower surfaces extend orthogonal to the second intermediate surface.

6. The endoscope according to claim 1, further comprising a spring-loaded connecting member extending from the first upper surface of the reusable first body, the spring-loaded connecting member is configured to be received in a pocket formed in the first lower surface of the disposable second body to inhibit the reusable first body and the disposable second body from separating from each other in a lateral direction.

7. The endoscope according to claim 6, wherein the angulation drive assembly includes:
a first pulley associated with the reusable first body and defining a first rotational axis; and
a second pulley associated with the disposable second body and defining a second rotational axis, the second pulley coupled to the first pulley such that rotation of the first pulley is transmitted to the second pulley,
wherein the first pulley and second pulley are coupled to each other at a location where the first intermediate surface abuts against the second intermediate surface.

8. The endoscope according to claim 7, further comprising a first electrical coupling associated with the reusable first body and a second electrical coupling associated with the disposable second body, and wherein the first and second electrical couplings are electrically coupled to each other at a location where the first intermediate surface abuts against the second intermediate surface.

9. The endoscope according to claim 7, further comprising a first electrical coupling associated with the reusable first body and a second electrical coupling associated with the disposable second body, and where the first and second electrical couplings are electrically coupled to each other at a location between the spring-loaded connecting member and the first and second pulleys.

10. The endoscope according to claim 1, wherein the angulation drive assembly includes:
a first pulley associated with the reusable first body and defining a first rotational axis; and
a second pulley associated with the disposable second body and defining a second rotational axis, the second pulley coupled to the first pulley such that rotation of the first pulley is transmitted to the second pulley,
wherein the first pulley and second pulley are coupled to each other at a location where the first intermediate surface abuts against the second intermediate surface.

11. The endoscope according to claim 1, wherein:
the reusable first body includes a first end wall that defines the first upper surface;
the disposable second body includes a second end wall that defines the first lower surface;
at least one window formed in one of the first end wall or the second end wall;
a first fiber bundle at least partially disposed within the reusable first body and having a first end electrically connected to an illumination source and a second end adjacent to the at least one window; and
a second fiber bundle at least partially disposed within the disposable second body and spaced apart from the first fiber bundle, the second fiber bundle having a third end adjacent to the at least one window and a fourth end associated with the insertion tube, the third end of the second fiber bundle being coaxial with the second end of the first fiber bundle so that light from the illumination source is transmitted from the first fiber bundle to the second fiber bundle.

12. The endoscope according to claim 11, wherein the first fiber bundle includes a first diameter and the second fiber bundle includes a second diameter, and wherein the first diameter is greater than the second diameter.

13. The endoscope according to claim 11, wherein the at least one window is made of a transparent material.

14. The endoscope according to claim 11, wherein the at least one window is formed in the first end wall.

15. The endoscope according to claim 11 further comprising a first window formed in the first end wall and a second window formed in the second end wall.

16. The endoscope according to claim 1, further comprising:
a female electrical connector formed in the first intermediate surface of the reusable first body; and
a male electrical connector extending from the second intermediate surface of the disposable second body, the male electrical connector configured to be received in the electrical female connector to electrically couple the female electrical connector and the male electrical connector to each other.

17. The endoscope according to claim 1, further comprising a handle assembly including:
a connecting member rotationally fixed to a pulley of the angulation drive assembly; and
a knob removably coupled to the connecting member, when the knob is coupled to the connecting member, rotation of the knob causes corresponding rotation of the connecting member and the pulley of the angulation drive assembly.

18. The endoscope according to claim 6, further comprising an attachment mechanism including a first attachment member associated with one of the reusable first body and the disposable second body and a second attachment member associated with the other of the reusable first body and the disposable second body, and wherein the first attachment member snaps into engagement with the second attachment member.

19. The endoscope according to claim 18, wherein the first attachment member is a flexible tab secured to the one of the reusable first body and the disposable second body and the second attachment member is a recess formed in the other of the reusable first body and the disposable second body.

20. The endoscope according to claim 18, wherein the attachment mechanism secures the first reusable body and the second disposable body to each other at a location diametrically opposite of the spring-loaded connecting member.

21. An endoscope comprising:
a control body including a first body and a second body removably coupled to the first body;
an insertion tube coupled to the control body;
a bending section secured to a distal end of the insertion tube; and
an angulation drive assembly coupled to the bending section and operable to articulate the bending section, the angulation drive assembly including:
a first pulley device associated with the first body and including a first pulley and a first transmission element, the first pulley defining a first rotational axis; and
a second pulley device associated with the second body and including a second pulley and a second transmission element operably connected to the bending section, the second pulley coupled to the first pulley such that rotation of the first pulley causes rotation of the second pulley, the second pulley also defining a second rotational axis,
wherein the first rotational axis of the first pulley and the second rotational axis of the second pulley are aligned with each other.

22. The endoscope according to claim 21, wherein the first body is reusable and the second body is disposable.

23. The endoscope according to claim 21, wherein the first transmission element is a belt and the second transmission element is one or more wires.

24. The endoscope according to claim 21, wherein the first transmission element is made of a first material and the second transmission element is made of a second material, the first material has a greater strength than the second material.

25. The endoscope according to claim 21, wherein:
the first pulley includes first teeth;
the first pulley device further includes a knob pulley that includes second teeth; and
the first transmission element transmits rotational motion of the knob pulley to the first pulley,
wherein there are at least twice as many second teeth than first teeth.

26. The endoscope according to claim 25, further comprising a handle assembly including:
a connecting member rotationally fixed to the knob pulley; and
a knob removably coupled to the connecting member at a location external to the control body, when the knob is coupled to the connecting member, rotation of the knob causes corresponding rotation of the connecting member and the knob pulley.

27. The endoscope according to claim 21, wherein:
the first pulley includes a central recess formed therein; and
the second pulley includes a central peg that corresponds to and is disposed within the central recess such that the first pulley and second pulley are rotationally fixed to each other.

28. The endoscope according to claim 21, wherein the second pulley device is located external relative to the first body.

29. The endoscope according to claim 21, wherein the first pulley comprises a first magnet and the second pulley comprises a second magnet, and wherein the first magnet and the second magnet are attracted to each other such that the second pulley and the first pulley are rotationally secured to each other when the first and second bodies are coupled to each other.

30. The endoscope according to claim 29, wherein, when the first and second bodies are coupled to each other, the first and second bodies define a compartment, the second magnet disposed within the compartment.
